SEQUENCE LISTING (83 sequences)

SEQ ID NO: 1 - DNA, 15 bp, Brassica napus
  source 1..15: mol_type = genomic DNA
cgcaacattgaccct

SEQ ID NO: 2 - DNA, 17 bp, Brassica napus
  source 1..17: mol_type = genomic DNA
acattgacccttgtcta

SEQ ID NO: 3 - DNA, 17 bp, Brassica napus
  source 1..17: mol_type = genomic DNA
ctcgcaacattgacctg

SEQ ID NO: 4 - DNA, 17 bp, Brassica napus
  source 1..17: mol_type = genomic DNA
tgtgcagacttttcaaa

SEQ ID NO: 5 - DNA, 17 bp, Brassica napus
  source 1..17: mol_type = genomic DNA
agtgtcttgtgcagact

SEQ ID NO: 6 - DNA, 16 bp, Brassica napus
  source 1..16: mol_type = genomic DNA
tgtcagcagcactaca

SEQ ID NO: 7 - DNA, 17 bp, Brassica napus
  source 1..17: mol_type = genomic DNA
agtcataccttttcagc

SEQ ID NO: 8 - DNA, 16 bp, Brassica napus
  source 1..16: mol_type = genomic DNA
tcgattggagtcatac

SEQ ID NO: 9 - DNA, 16 bp, Brassica napus
  source 1..16: mol_type = genomic DNA
ccttttcagcatgttt

SEQ ID NO: 10 - DNA, 16 bp, Brassica napus
  source 1..16: mol_type = genomic DNA
taaggtgcacgtgact

SEQ ID NO: 11 - DNA, 16 bp, Brassica napus
  source 1..16: mol_type = genomic DNA
ctcggacataattaat

SEQ ID NO: 12 - DNA, 16 bp, Brassica napus
  source 1..16: mol_type = genomic DNA
acgtgactcggacata

SEQ ID NO: 13 - DNA, 20 bp, Brassica napus
  source 1..20: mol_type = genomic DNA
actccaacgaaacagcaagg

SEQ ID NO: 14 - DNA, 19 bp, Brassica napus
  source 1..19: mol_type = genomic DNA
ccaacgaaacagcaaggaa

SEQ ID NO: 15 - DNA, 21 bp, Brassica napus
  source 1..21: mol_type = genomic DNA
aaaactccaacgaaacagcaa

SEQ ID NO: 16 - DNA, 20 bp, Brassica napus
  source 1..20: mol_type = genomic DNA
tttcttgcagaggaggagga

SEQ ID NO: 17 - DNA, 20 bp, Brassica napus
  source 1..20: mol_type = genomic DNA
ttcttgcagaggaggaggag

SEQ ID NO: 18 - DNA, 20 bp, Brassica napus
  source 1..20: mol_type = genomic DNA
tcttgcagaggaggaggagt

SEQ ID NO: 19 - DNA, 24 bp, Brassica napus
  source 1..24: mol_type = genomic DNA
ttttcataggtgaagttgggagat

SEQ ID NO: 20 - DNA, 22 bp, Brassica napus
  source 1..22: mol_type = genomic DNA
tttcataggtgaagttgggaga

SEQ ID NO: 21 - DNA, 21 bp, Brassica napus
  source 1..21: mol_type = genomic DNA
ttcataggtgaagttgggaga

SEQ ID NO: 22 - DNA, 20 bp, Brassica napus
  source 1..20: mol_type = genomic DNA
ggtttgctgacatggaggat

SEQ ID NO: 23 - DNA, 20 bp, Brassica napus
  source 1..20: mol_type = genomic DNA
aggtttgctgacatggagga

SEQ ID NO: 24 - DNA, 20 bp, Brassica napus
  source 1..20: mol_type = genomic DNA
caaaggtttgctgacatgga

SEQ ID NO: 25 - DNA, 20 bp, Brassica napus
  source 1..20: mol_type = genomic DNA
tcctcctcctctgcaagaaa

SEQ ID NO: 26 - DNA, 20 bp, Brassica napus
  source 1..20: mol_type = genomic DNA
cctcctcctctgcaagaaaa

SEQ ID NO: 27 - DNA, 20 bp, Brassica napus
  source 1..20: mol_type = genomic DNA
tgactcctcctcctctgcaa

SEQ ID NO: 28 - DNA, 20 bp, Brassica napus
  source 1..20: mol_type = genomic DNA
gtttggggtttggcctactt

SEQ ID NO: 29 - DNA, 19 bp, Brassica napus
  source 1..19: mol_type = genomic DNA
tttggggtttggcctactt

SEQ ID NO: 30 - DNA, 20 bp, Brassica napus
  source 1..20: mol_type = genomic DNA
acttggcatgtagtgctgct

SEQ ID NO: 31 - DNA, 20 bp, Brassica napus
  source 1..20: mol_type = genomic DNA
cgacgtcctgctcctgttat

SEQ ID NO: 32 - DNA, 21 bp, Brassica napus
  source 1..21: mol_type = genomic DNA
acgtcctgctcctgttattca

SEQ ID NO: 33 - DNA, 20 bp, Brassica napus
  source 1..20: mol_type = genomic DNA
acgacgtcctgctcctgtta

SEQ ID NO: 34 - DNA, 20 bp, Brassica napus
  source 1..20: mol_type = genomic DNA
ccgaattatttaggcggtga

SEQ ID NO: 35 - DNA, 21 bp, Brassica napus
  source 1..21: mol_type = genomic DNA
ccgaattatttaggcggtgat

SEQ ID NO: 36 - DNA, 21 bp, Brassica napus
  source 1..21: mol_type = genomic DNA
gccgaattatttaggcggtga

SEQ ID NO: 37 - DNA, 16 bp, Brassica napus
  source 1..16: mol_type = genomic DNA
tgctgtatctgctttt

SEQ ID NO: 38 - DNA, 15 bp, Brassica napus
  source 1..15: mol_type = genomic DNA
atgtgctgtatctgc

SEQ ID NO: 39 - DNA, 18 bp, Brassica napus
  source 1..18: mol_type = genomic DNA
tttgctatgtgctgtatc

SEQ ID NO: 40 - DNA, 17 bp, Brassica napus
  source 1..17: mol_type = genomic DNA
agtcataccttttcagc

SEQ ID NO: 41 - DNA, 16 bp, Brassica napus
  source 1..16: mol_type = genomic DNA
tcgattggagtcatac

SEQ ID NO: 42 - DNA, 16 bp, Brassica napus
  source 1..16: mol_type = genomic DNA
ccttttcagcatgttt

SEQ ID NO: 43 - DNA, 16 bp, Brassica napus
  source 1..16: mol_type = genomic DNA
taaggtgcacgtgact

SEQ ID NO: 44 - DNA, 16 bp, Brassica napus
  source 1..16: mol_type = genomic DNA
ctcggacataattaat

SEQ ID NO: 45 - DNA, 16 bp, Brassica napus
  source 1..16: mol_type = genomic DNA
acgtgactcggacata

SEQ ID NO: 46 - DNA, 22 bp, Brassica napus
  source 1..22: mol_type = genomic DNA
gccgtttgctagtcaagctagt

SEQ ID NO: 47 - DNA, 22 bp, Brassica napus
  source 1..22: mol_type = genomic DNA
cgtttgctagtcaagctagtcg

SEQ ID NO: 48 - DNA, 23 bp, Brassica napus
  source 1..23: mol_type = genomic DNA
agccgtttgctagtcaagctagt

SEQ ID NO: 49 - DNA, 24 bp, Brassica napus
  source 1..24: mol_type = genomic DNA
ttttcataggtgaagttgggagat

SEQ ID NO: 50 - DNA, 22 bp, Brassica napus
  source 1..22: mol_type = genomic DNA
tttcataggtgaagttgggaga

SEQ ID NO: 51 - DNA, 21 bp, Brassica napus
  source 1..21: mol_type = genomic DNA
ttcataggtgaagttgggaga

SEQ ID NO: 52 - DNA, 20 bp, Brassica napus
  source 1..20: mol_type = genomic DNA
ggtttgctgacatggaggat

SEQ ID NO: 53 - DNA, 20 bp, Brassica napus
  source 1..20: mol_type = genomic DNA
aggtttgctgacatggagga

SEQ ID NO: 54 - DNA, 20 bp, Brassica napus
  source 1..20: mol_type = genomic DNA
caaaggtttgctgacatgga

SEQ ID NO: 55 - DNA, 20 bp, Brassica napus
  source 1..20: mol_type = genomic DNA
caaaggtttgctgacatgga

SEQ ID NO: 56 - DNA, 21 bp, Brassica napus
  source 1..21: mol_type = genomic DNA
ttgtcctgggtcttcttgaca

SEQ ID NO: 57 - DNA, 21 bp, Brassica napus
  source 1..21: mol_type = genomic DNA
tctttgtcctgggtcttcttg

SEQ ID NO: 58 - DNA, 20 bp, Brassica napus
  source 1..20: mol_type = genomic DNA
cgacgtcctgctcctgttat

SEQ ID NO: 59 - DNA, 21 bp, Brassica napus
  source 1..21: mol_type = genomic DNA
acgtcctgctcctgttattca

SEQ ID NO: 60 - DNA, 20 bp, Brassica napus
  source 1..20: mol_type = genomic DNA
acgacgtcctgctcctgtta

SEQ ID NO: 61 - DNA, 20 bp, Brassica napus
  source 1..20: mol_type = genomic DNA
ccgaattatttaggcggtga

SEQ ID NO: 62 - DNA, 21 bp, Brassica napus
  source 1..21: mol_type = genomic DNA
ccgaattatttaggcggtgat

SEQ ID NO: 63 - DNA, 21 bp, Brassica napus
  source 1..21: mol_type = genomic DNA
gccgaattatttaggcggtga

SEQ ID NO: 64 - DNA, 50 bp, Brassica napus
  source 1..50: mol_type = genomic DNA
ggctcttgaacacatcgggtttcgatatgacgaacatgatctgtgatgtt

SEQ ID NO: 65 - DNA, 50 bp, Brassica napus
  source 1..50: mol_type = genomic DNA
ttctgaatgcagatggctctagcgtgattataacaggcgtgagatacatg

SEQ ID NO: 66 - DNA, 50 bp, Brassica napus
  source 1..50: mol_type = genomic DNA
acgagagacataatagtgggtgagaattgatgatctgagctctctgactt

SEQ ID NO: 67 - DNA, 50 bp, Brassica napus
  source 1..50: mol_type = genomic DNA
catcaattcaaacatctactccctccgttttataaagagtgtcacttaaa

SEQ ID NO: 68 - DNA, 50 bp, Brassica napus
  source 1..50: mol_type = genomic DNA
ggcatggtgaaaaaaggttcagattctgtcacaaaagattacagagataa

SEQ ID NO: 69 - DNA, 50 bp, Brassica napus
  source 1..50: mol_type = genomic DNA
agatccatcccagctgccggagaagccagcggaatgttgatttttcgatc

SEQ ID NO: 70 - DNA, 50 bp, Brassica napus
  source 1..50: mol_type = genomic DNA
tactcgtcccttggtttttgcttcttttttcctggttctgtattgaacct

SEQ ID NO: 71 - DNA, 50 bp, Brassica napus
  source 1..50: mol_type = genomic DNA
atctaaagcaggagctggttttgcaaaatgtggtacagttcaagagactt

SEQ ID NO: 72 - DNA, 50 bp, Brassica napus
  source 1..50: mol_type = genomic DNA
agacggttgttaggagaattctataatgtgttgttgcacaaccatatgcc

SEQ ID NO: 73 - DNA, 50 bp, Brassica napus
  source 1..50: mol_type = genomic DNA
ataatactgaacaaggaggaacaggtgaacaagagtagtcagaacacggc

SEQ ID NO: 74 - DNA, 50 bp, Brassica napus
  source 1..50: mol_type = genomic DNA
tacttctatgttatgaaggatcggataacccctaaaccctaaaaacaggg

SEQ ID NO: 75 - DNA, 50 bp, Brassica napus
  source 1..50: mol_type = genomic DNA
caggacaaaggagagaggtatccgtttgatttttggtgttccgataatcc

SEQ ID NO: 76 - DNA, 50 bp, Brassica napus
  source 1..50: mol_type = genomic DNA
aatgaagtgaggaatgtgctatgagtattgtggtggttgatgttgaagag

SEQ ID NO: 77 - DNA, 50 bp, Brassica napus
  source 1..50: mol_type = genomic DNA
cctgctcctgttattcataaacatgctgaaaaggtatgactccaatcgac

SEQ ID NO: 78 - DNA, 50 bp, Brassica napus
  source 1..50: mol_type = genomic DNA
gccattttgttcctccttcgatgctcgtggacagagactggaagaaaaaa

SEQ ID NO: 79 - DNA, 50 bp, Brassica napus
  source 1..50: mol_type = genomic DNA
cttctaacaagagtcttccaaatctggcctgagtaacgacatccaaagaa

SEQ ID NO: 80 - DNA, 201 bp, Brassica napus
  source 1..201: mol_type = genomic DNA
ctctatcatcttggtcgctgctctgatgacttcgatttgtggtcctagccattgtggtgacgtccttagcgcgtacctgcagcttaaaagaaaagaaaaaagtagctaaccaagccaaaattcaaacttcagtttagaaattcagttcggttatttcggatattagataattcagttaagaggattatagtatccactgtt

SEQ ID NO: 81 - DNA, 201 bp, Brassica napus
  source 1..201: mol_type = genomic DNA
tcaaattacatacacacaaaaaaaaattttgtggttttgtactaatattattacttaactttacaaatataaacaaatttaaatcagtttgcgctgatttaaactaatttaaaatggtttaaatcatttgaattgaatattgtttttcctagggtcgatttgccacttagccggacacccctagaccaaatctttagaaca

SEQ ID NO: 82 - DNA, 201 bp, Brassica napus
  source 1..201: mol_type = genomic DNA
gtgcctaatgtcagcaaacttttatttatatatgatccttccttgcgtcttaaaccactggtttcaacctttgaactctcggaacctgccagatcaacgagattctgcaggtacggaagagattatgggtagttaatgtcagttgctcttagtgttgaagaaagagttgatcataaccatacataatatcacagacacaag

SEQ ID NO: 83 - DNA, 201 bp, Brassica napus
  source 1..201: mol_type = genomic DNA
atctgttgacattgacaatcgaaagcatagttaaggtgcataaaatcttttttccaaatacttaatcttcttcatttttgacgtacagtattattgtcgatagttgatgtattattttaataagtttcaattattcaaagtcgagggaatgttgcaaaatcatgccaattattattttgaacacagttgtcctttgcgaca

(19) United States
(12) Patent Application Publication
(10) Pub. No.: US 2026/0337699 A1
(43) Pub. Date: Oct. 29, 2026

(54) N15 DELETION QTL FOR LOW FIBER, COMPOSITIONS AND METHODS IN BRASSICA

(71) Applicant: PIONEER HI-BRED INTERNATIONAL, INC., JOHNSTON, IA (US)

(72) Inventors: SARAH ATWOOD, URBANDALE, IA (US); IGOR FALAK, GUELPH, IA (US); KEVIN A. FENGLER, CLIVE, IA (US); XIUQIANG HUANG, MISSISSUAGA, IA (US); SIVA S. AMMIRAJU JETTY, JOHNSTON, IA (US); SCOTT CHARLES JOBGEN, WEST DES MOINES, IA (US)

(21) Appl. No.: 19/117,816

(22) PCT Filed: Oct. 3, 2023
(86) PCT No.: PCT/US2023/075784

§ 371 Date: Apr. 2, 2025

Related U.S. Application Data
(60) Provisional application No. 63/378,320, filed on Oct. 4, 2022.

(51) Int. Cl. C12Q 1/6895; A01H 1/00; A01H 1/04; A23K 10/37

(52) U.S. Cl. CPC C12Q 1/6895; A01H 1/045; A01H 1/10; A23K 10/37; C12Q 2600/13; C12Q 2600/156

(57) ABSTRACT

This disclosure concerns methods and compositions for identifying canola plants that have a low fiber content trait. Some embodiments concern a quantitative trait locus and chromosomal segment deletion which are associated with low fiber content in canola plants, germplasm, and meal. Some embodiments concern methods and molecular markers to identify, select, and/or construct low fiber content canola plants and germplasm. In some embodiments, methods and molecular markers are provided to select or to identify and counter-select plants with relatively higher fiber content. This disclosure also concerns canola plants comprising a low fiber content trait that are generated by methods utilizing at least one marker described herein.

### REFERENCE TO SEQUENCE LISTING SUBMITTED ELECTRONICALLY

The official copy of the sequence listing is submitted concurrently with the specification as an XML formatted sequence listing with a file named 108398-WO-SEC_ST.26 created on Oct. 2, 2023 and having a size of 74,739 bytes. This sequence listing is part of the specification and is herein incorporated by reference in its entirety.

### TECHNICAL FIELD

This disclosure relates to the discovery of a quantitative trait locus (QTL) associated with a low fiber content trait in canola (Brassica napus); provided are methods and compositions for identifying the low fiber content trait in canola as well as methods for breeding with, selecting, introgressing, and/or introducing the low fiber content QTL trait into canola.

### BACKGROUND

Canola or oilseed rape (Brassica napus L., 2n=4x=38, AACC), an allotetraploid formed from diploids B. rapa (2n=2x=20, AA) and B. oleracea (2n=2x=18, CC), is one of the most important vegetable oilseed crops in the world, especially in China, Canada, the European Union and Australia. Canola meal, the fraction of the seed remaining after crushing and oil extraction, is approximately 55% of the volume of canola seed.

Canola meal consists of several components including protein, fiber, residual oil, carbohydrates, and anti-nutritional factors. Although canola meal is relatively high in protein, its high fiber content decreases its digestibility and its value as an animal feed. Compared to soybean meal, canola meal contains higher values of dietary fiber and a lower percentage of protein. Because of its high dietary fiber, canola meal has about 20% less metabolizable energy (ME) than soybean meal. As a result, the value of the meal has remained low relative to other oilseed meals such as soybean meal, particularly in rations for pigs and poultry. Rakow (2004a) Canola meal quality improvement through the breeding of yellow-seeded varieties—an historical perspective, in AAFC Sustainable Production Systems Bulletin. Additionally, the presence of glucosinolates in some canola meals also decreases its value, due to the deleterious effects these compounds have on the growth and reproduction of livestock.

Canola varieties are distinguished in part by their seed coat color. Seed coat color is generally divided into two main classes: yellow and black (or dark brown). Varying shades of these colors, such as reddish brown and yellowish brown, are also observed. Canola varieties with lighter seed coat color have been widely observed to have thinner hulls, and thus less fiber and more oil and protein than varieties with dark color seed coats. Stringam et al. (1974) Chemical and morphological characteristics associated with seed coat color in rapeseed, in Proceedings of the 4th International Rapeseed Congress, Giessen, Germany, pp. 99-108; Bell and Shires (1982) Can. J. Animal Science 62:557-65; Shirzadegan and Röbbelen (1985) Götingen Fette Seifen Anstrichmittel 87:235-7; Simbaya et al. (1995) J. Agr. Food Chem. 43:2062-6; Rakow (2004b) Yellow-seeded Brassica napus canola for the Canadian canola industry, in AAFC Sustainable Production Systems Bulletin. One possible explanation for this is that the canola plant may expend more energy into the production of proteins and oils if it does not require that energy for the production of seed coat fiber components. Yellow-seeded canola lines also have been reported to have lower glucosinolate content than black-seeded canola lines. Rakow et al. (1999b) Proc. 10th Int. Rapeseed Congress, Canberra, Australia, Sep. 26-29, 1999, Poster #9. Thus, historically the development of yellow-seeded canola varieties has been pursued as a potential way to increase the feed value of canola meal. Bell (1995) Meal and by-product utilization in animal nutrition, in Brassica oilseeds, production and utilization. Eds. Kimber and McGregor, Cab International, Wallingford, Oxon, OX108DE, UK, pp. 301-37; Rakow (2004b), supra; Rakow & Raney (2003).

Some yellow-seeded forms of Brassica species closely related to B. napus (e.g., B. rapa and B. juncea) have been shown to have lower levels of fiber in their seed and subsequent meal. Scientists at Agriculture and Agri-Foods Canada (AAFC) have developed yellow seed coat (YSC) lines (YN86-37, YN90-1016, YN97-262 and YN01-429) of low hull proportion with thinner seed coat, low fiber and high oil compared to the black seed coat (BSC) canola (Rakow et al., 2011). Feeding studies, comparing yellow seeded canola meal from AAFC line YN01-429 to B. juncea, B. rapa, and brown-seeded B. napus, demonstrated the advantages of YSC B. napus line such as higher protein, lower fiber, increased amino acid digestibility and metabolizable energy content, and improved nutrient and energy utilization based on feed to gain ratio in broiler chickens and monogastric animal species (Hickling, 2009; Slominski et al., 2010).

The development of yellow-seeded B. napus germplasm has demonstrated that fiber can be reduced in B. napus through the integration of genes controlling seed pigmentation from related Brassica species. However, the breeding of low fiber content has been greatly hampered by a poor understanding of the inheritance and stability of the low fiber content traits, as well as a lack of robust, high-throughput markers tightly linked to the trait. Due to allotetraploidy, effect of multiple genes, maternal effects and environmental effects, the inheritance of low fiber content trait is complex, and fine mapping studies/identification of markers tightly linked to this trait is very challenging. Traditional techniques for selection of lower fiber canola lines have primarily relied on fiber content data obtained using cost and labor-intensive analytical methods, or seed coat color, because of its high correlation with low fiber in the AAFC YSC lines.

Comparatively little information is available regarding the extent of variability of fiber content there is within dark-seeded B. napus germplasm. Limited reports have been made of dark-seeded canola lines having been developed that contain reduced levels of anti-nutritional factors (e.g., fiber and polyphenolic compounds), and increased protein levels. See e.g., U.S. Pat. Nos. 9,596,871, and 10,791,692 and International Application Publication No. WO 2020/131600. These desirable nutritional characteristics make this germplasm particularly valuable as sources for canola meal. However, few molecular markers that are tightly linked to this desirable nutritional trait in dark-seeded canola lines have been previously described.

### BRIEF SUMMARY OF THE DISCLOSURE

The methods, assays and molecular markers provided herein are based, at least in part, on the discovery of unexpected deletions of a genomic region or quantitative trait locus (“QTL”) in Brassica chromosome N15 (“N15”) that can provide desirable nutritional traits, including low fiber content in Brassica. Described herein are methods, probes, primers, assays and molecular markers for identifying this QTL which is associated with low fiber content. Thus the disclosed methods, assays and molecular markers can be used to identify plant material (the term “plant material” is used herein tor refer to any of a plant, cell, seed, tissue, or germplasm thereof) having (i) reduced fiber content relative to sibling plants or isogenic plants lacking the disclosed QTL on N15 and/or (ii) genetics suitable for use in a breeding program to generate progeny plants having the reduced fiber phenotype associated with the QTL on N15. The term “reduced fiber” is used herein to mean the lower fiber content of plant having an N15 QTL deletion as compared to an isogenic plant, parent plant, or sibling progeny plant lacking the N15 QTL deletion. The methods, assays, and molecular markers can be used with a Brassica crop plant. As used herein, Brassica preferably refers to Brassica napus, Brassica juncea, Brassica carinata, Brassica rapa or Brassica oleracea.

In one aspect, a method is provided that includes providing or isolating a sample comprising nucleic acid from Brassica plant material and screening the sample for a deleted genomic segment on chromosome N15, wherein the deleted segment is located between genomic sequences SEQ ID NO:65 (N101HTV-001) and SEQ ID NO:78 (N101HGY-001) and the N15 deletion is associated with low fiber content in Brassica napus. For example, the disclosed method can include screening the sample for the absence of one or more genomic markers between position 86.09 cM and position 101.53 cM of chromosome N15, e.g., between position 97.93 cM and position 101.24 cM of chromosome N15. The disclosed method can include screening the sample for the absence of one or more genomic markers between physical nucleotide positions from about 51,500,326 to about 53,229,532 in chromosome N15; e.g., from about physical nucleotide positions 52,639,876 to about 53,229,532 or from about physical nucleotide positions 51,500,326 to about 53,185,850 in publicly available Brassica napus reference genome for Darmor V10. See Rousseau-Gueutin et al., 2020, Gigascience 9(12) giaa137. 10.1093/gigascience/giaa137. The method can include screening the sample for such an N15 deletion, wherein the deleted segment is at least about 500 kb in length, at least about 532 kb in length, at least about 575 kb in length, at least about 600 kb in length, at least about 700 kb in length, at least about 800 kb in length, at least about 900 kb in length, at least about 1,000 kb in length, or at least about 1,667 kb in length. In some examples, the method can also include screening the sample for the presence of a heterologous insertion sequence, i.e., an insertion that has replaced the deleted genomic segment on chromosome N15.

The provided methods can include isolating or providing a sample comprising nucleic acid from Brassica plant material and screening the sample for a disclosed deleted genomic segment on chromosome N15. Screening for a deletion can be done by nucleotide sequencing and/or by any other suitable method for detecting a genetic polymorphisms, which include methods for marker detection and marker assisted selection methods, disclosed herein. Thus, the method can include using sequencing or a method for detecting a genetic polymorphisms to screen for a deleted segment of genomic sequence between genomic positions that correspond to SEQ ID NO:65 (N101HTV-001) and SEQ ID NO:78 (N101HGY-001). In some examples, the deletion segment is a deletion of sequence between the breakpoints identified herein, e.g., within genomic positions that correspond to SEQ ID NO:82 and SEQ ID NO:81; or within genomic positions that correspond to SEQ ID NO:82 and SEQ ID NO:83; or within genomic positions that correspond to SEQ ID NO:80 and SEQ ID NO:83; or within genomic positions that correspond to SEQ ID NO:80 and SEQ ID NO:81. The absence of such genomic sequence(s) or marker alleles is indicative of a deleted segment of chromosome N15. Additionally or alternatively, the disclosed method can include detecting the presence of a heterologous insertion sequence that has replaced the deleted genomic segment on chromosome N15.

The method of identifying Brassica plant material comprising a deleted segment can include screening the sample for the absence of about 580,000 nucleotides which are present in wild type N15 chromosome, wherein the method comprises using one or more of the wildtype probe or marker sequences disclosed in Table 7 herein (SEQ ID NO:1 through 79). Alternatively or additionally, the method can include screening the sample for the presence of one or more of the deletion probe or marker sequences disclosed in Table 7 herein to further determine the zygosity of the N15 QTL in the sample. Screening for the presence or absence of these sequences can be done using any suitable method for detecting a polymorphism, including such methods disclosed herein.

The method of identifying Brassica plant material comprising a deleted segment can include screening the sample for the absence of about 1,670,000 nucleotides which are present in wild type N15 chromosome. For example, the method can include screening the sample for the presence or absence of one or more genomic sequence having 90%, 91%, 92%, 93%, 94%, 95%, 96%, 97%, 98%, 99%, or 100% sequence identity to one or more of SEQ ID NOs:66 through 78, wherein the absence of the sequence indicates the plant material comprises the deleted segment. Screening for the presence or absence of these sequences can be done using any suitable method for detecting a polymorphism, including such methods disclosed herein.

In particular examples, the disclosed method for identifying plant material includes providing or isolating a nucleic acid sample from Brassica napus plant material and screening the sample for a nucleic acid that comprises one or more chromosome N15 deletion segment using (I) a nucleic acid probe comprising 90%, 91%, 92%, 93%, 94%, 95%, 96%, 97%, 98%, 99%, or 100% sequence identity to one or more of SEQ ID NO:1, SEQ ID NO:2, SEQ ID NO:3, SEQ ID NO:4, SEQ ID NO:5, SEQ ID NO:6, or a combination of 2 or more, 3 or more, 4 or more, 5, or 6 of the foregoing probes; (II) nucleic acid forward primer comprising 90%, 91%, 92%, 93%, 94%, 95%, 96%, 97%, 98%, 99%, or 100% sequence identity to one or more of SEQ ID NO:13, SEQ ID NO:14, SEQ ID NO:15, SEQ ID NO:16, SEQ ID NO:17, or SEQ ID NO:18 and a nucleic acid reverse primer comprising 90%, 91%, 92%, 93%, 94%, 95%, 96%, 97%, 98%, 99%, or 100% sequence identity to one or more of SEQ ID NO:25, SEQ ID NO:26, SEQ ID NO:27, SEQ ID NO:28, SEQ ID NO:29, or SEQ ID NO:30, including any combination of the foregoing forward and reverse primers; (III) a nucleic acid probe comprising 90%, 91%, 92%, 93%, 94%, 95%, 96%, 97%, 98%, 99%, or 100% sequence identity to one or more of SEQ ID NO:37, SEQ ID NO:38, SEQ ID NO:39, or any combination of 2 or 3 probes thereof; or (IV) a nucleic acid forward primer comprising 90%, 91%, 92%, 93%, 94%, 95%, 96%, 97%, 98%, 99%, or 100% sequence identity to one or more of SEQ ID NO:46, SEQ ID NO:47, or SEQ ID NO:48, and a nucleic acid reverse primer comprising 90%, 91%, 92%, 93%, 94%, 95%, 96%, 97%, 98%, 99%, or 100% sequence identity to one or more of SEQ ID NO:55; SEQ ID NO:56, or SEQ ID NO:57, including any combination of the foregoing forward and reverse primers. In some examples, this of method screening the sample for a nucleic acid that comprises one or more chromosome N15 deletion segment can include (or can further include) screening for zygosity of the deletion segment by screening for the presence of the corresponding wild type alleles (indicating the absence of the N15 deletion on one copy of chromosome 15) using (I) a nucleic acid probe comprising 90%, 91%, 92%, 93%, 94%, 95%, 96%, 97%, 98%, 99%, or 100% sequence identity to one or more of SEQ ID NO:7, SEQ ID NO:8, SEQ ID NO:9, SEQ ID NO:10, SEQ ID NO:11, or SEQ ID NO:12, or SEQ ID NO:40, SEQ ID NO:41, SEQ ID NO:42, SEQ ID NO:43, SEQ ID NO:44, or SEQ ID NO:45 or a combination of 2 or more, 3 or more, 4 or more, 5 or more, 6 or more, 7 or more, 8 or more, 9 or more, 10 or more, 11 or 12 of the foregoing probes; (II) nucleic acid forward primer comprising 90%, 91%, 92%, 93%, 94%, 95%, 96%, 97%, 98%, 99%, or 100% sequence identity to one or more of SEQ ID NO:19, SEQ ID NO:20, SEQ ID NO.21, SEQ ID NO:22, SEQ ID NO:23, or SEQ ID NO:24, and a nucleic acid reverse primer comprising 90%, 91%, 92%, 93%, 94%, 95%, 96%, 97%, 98%, 99%, or 100% sequence identity to one or more of SEQ ID NO:31, SEQ ID NO:32, SEQ ID NO:33, SEQ ID NO:34, SEQ ID NO:35, or SEQ ID NO:36 including any combination of the foregoing forward and reverse primers; or (III) nucleic acid forward primer comprising 90%, 91%, 92%, 93%, 94%, 95%, 96%, 97%, 98%, 99%, or 100% sequence identity to one or more of SEQ ID NO:49, SEQ ID NO:50, EQ ID NO:51, SEQ ID NO:52, SEQ ID NO:53, or SEQ ID NO:54, and a nucleic acid reverse primer comprising 90%, 91%, 92%, 93%, 94%, 95%, 96%, 97%, 98%, 99%, or 100% sequence identity to one or more of SEQ ID NO:58, SEQ ID NO:59, SEQ ID NO:60; SEQ ID NO:61, SEQ ID NO:62, or SEQ ID NO:63, including any combination of the foregoing forward and reverse primers.

The disclosed method for identifying plant material can include providing or isolating a nucleic acid sample from Brassica napus plant material and screening the sample for a nucleic acid that comprises one or more chromosome N15 deletion segment using a nucleic acid probe comprising 90%, 91%, 92%, 93%, 94%, 95%, 96%, 97%, 98%, 99%, or 100% sequence identity to one or more of SEQ ID NO:4. Optionally, the probe comprising SEQ ID NO:4 can be used to detect a PCR amplification product that is amplified using a forward primer comprising SEQ ID NO:16 and a reverse primer comprising SEQ ID NO:28. This method can further include screening for zygosity, e.g., screening for wild type N15 chromosome segment, e.g., using a nucleic acid probe comprising SEQ ID NO:7. Optionally, the probe comprising SEQ ID NO:7 can be used to detect a PCR amplification product that is amplified using a forward primer comprising SEQ ID NO:19 and a reverse primer comprising SEQ ID NO:31.

Thus, the screening of a sample for a nucleic acid comprising the N15 deletion QTL can be done using techniques such as allele-specific polymerase chain reaction (PCR) amplification or nucleic acid sequencing. For example, the sample can be screened by amplifying and/or sequencing from a position that is 10 to 300 bases upstream or downstream of any N15 deleted segment breakpoint identified herein to thereby detect an N15 QTL deletion, e.g., by detecting the absence of wild-type sequence within the deleted segment or by detecting the presence of insertion sequence that replaced the wild-type sequence. This screening method can further involve screening in the same manner a control sample that does not have the N15 QTL deletion (i.e., the N15 deleted segment is present/not deleted in the control sample).

This disclosure provides seed from a Brassica napus plant identified by a method disclosed herein as having one or more chromosome N15 deletion segment disclosed herein. This disclosure further provides meal made from such seed of a plant identified as having the one or more copies of the chromosome N15 deletion segment disclosed herein.

Also provided herein is a method for selecting one or more plants or germplasm thereof, from a population, wherein the selected plant or germplasm thereof comprises a quantitative trait locus (QTL) associated with desirable nutritional traits, including low fiber content in canola. The method includes obtaining or isolating a sample comprising nucleic acid from each plant or germplasm thereof, which are from a plurality of plants within a population of Brassica plants, screening each sample for a chromosome N15 comprising the deleted genomic segment disclosed herein. The screening method can be any of those disclosed herein, wherein the method further includes selecting one or more plants or germplasm thereof which are identified by the screening step as having a deleted genomic segment on chromosome N15. For example, the method includes screening each sample from a plant or germplasm in a plurality of plants or germplasm thereof using a method disclosed herein and selecting one or more plants or germplasm that provided a sample identified as having a deleted genomic segment on chromosome N15 located between genomic sequences corresponding to SEQ ID NO:65 (N101HTV-001) and SEQ ID NO:78 (N101HGY-001). Thus, the method of selecting plant material can include selecting plant or germplasm thereof whose sample is identified as (e.g., selected using a nucleic acid probe) comprising (I) SEQ ID NO:1, SEQ ID NO:2, SEQ ID NO:3, SEQ ID NO:4, SEQ ID NO:5, SEQ ID NO:6, or a combination of probes comprising 2 or more, 3 or more, 4 or more, 5 or 6 of the foregoing sequences or (II) SEQ ID NO:37, SEQ ID NO:38, SEQ ID NO:39, or any combination of 2 or 3 of the foregoing sequences. In particular examples, the method of selection involves screening each sample from the population using (I) nucleic acid forward primer comprising SEQ ID NO:13, SEQ ID NO:14, SEQ ID NO:15, SEQ ID NO:16, SEQ ID NO:17, or SEQ ID NO:18 and a nucleic acid reverse primer comprising SEQ ID NO:25, SEQ ID NO:26, SEQ ID NO:27, SEQ ID NO:28, SEQ ID NO:29, or SEQ ID NO:30, including any combination of these forward and reverse primers; (II) a nucleic acid forward primer comprising SEQ ID NO:46, SEQ ID NO:47, or SEQ ID NO:48, and a nucleic acid reverse primer comprising SEQ ID NO:55; SEQ ID NO:56, or SEQ ID NO:57, including any combination of these forward and reverse primers. In certain examples, the method of selection includes obtaining nucleic acid samples from a Brassica plant or germplasm, screening samples for and selecting at least one plant or germplasm thereof whose sample is identified as having sequence (e.g., selected using a nucleic acid probe) comprising SEQ ID NO:4. Optionally, the selected plant(s) or germplasm thereof are identified using a probe comprising SEQ ID NO:4 to detect a PCR amplification product that is amplified using a forward primer comprising SEQ ID NO:16 and a reverse primer comprising SEQ ID NO:28. This method can further include screening for zygosity, e.g., screening for wild type N15 chromosome segment, e.g., using a nucleic acid probe comprising SEQ ID NO:7 and counter-selecting plant material that is homozygous for wild type N15 chromosome segment comprising SEQ ID NO:7. Optionally, the probe comprising SEQ ID NO:7 can be used to detect a PCR amplification product that is amplified using a forward primer comprising SEQ ID NO:19 and a reverse primer comprising SEQ ID NO:31.

Moreover, each of the disclosed methods of selecting can be used to screen one or more plants, or germplasm thereof, in a plurality of plants within a population of Brassica plants, the step of screening each sample for a nucleic acid comprising one or more low fiber content marker alleles can be done using techniques such as allele-specific polymerase chain reaction (PCR) amplification or nucleic acid sequencing. For example, the sample can be screened by amplifying and/or sequencing from a position that is 10 to 300 bases upstream or downstream of an N15 deleted segment breakpoint to thereby detect an N15 QTL deletion (e.g., by detecting the absence of wild-type sequence within the deleted segment or by detecting the presence of insertion sequence that replaced the wild-type sequence) and the method then includes selecting one or more plants, or germplasm thereof, comprising the N15 QTL deletion.

In another aspect, a method is provided to produce a Brassica plant comprising the N15 deletion QTL disclosed herein. The method includes crossing a first plant having the N15 deletion QTL with a second plant lacking the N15 deletion QTL to produce progeny plants. Progeny plants are then screened in accordance with a method disclosed herein, e.g., by screening a sample from each of a plurality of progeny plants to identify and then select at least one progeny plant having the screened-for N15 deletion. In certain examples, a method of producing a Brassica plant comprises obtaining samples from each progeny in a plurality of progeny plants and screening each sample for a nucleic acid sample comprising a deleted genomic segment on chromosome N15 located between sequences SEQ ID NO:65 (N101HTV-001) and SEQ ID NO:78 (N101HGY-001). Thus, the method of selecting progeny plants can include screening each progeny plant sample for sequence (e.g., using a nucleic acid probe) comprising (I) SEQ ID NO:1, SEQ ID NO:2, SEQ ID NO:3, SEQ ID NO:4, SEQ ID NO:5, or SEQ ID NO:6 or a combination of probes comprising 2 or more, 3 or more, 4 or more, 5 or 6 of the foregoing sequences or (II) SEQ ID NO:37, SEQ ID NO:38, SEQ ID NO:39, or any combination of 2 or 3 of the foregoing sequences. In particular examples, the method of producing a Brassica plant comprises screening each progeny plant sample from the population using (I) nucleic acid forward primer comprising SEQ ID NO:13, SEQ ID NO:14, SEQ ID NO:15, SEQ ID NO:16, SEQ ID NO:17, or SEQ ID NO:18 and a nucleic acid reverse primer comprising SEQ ID NO:25, SEQ ID NO:26, SEQ ID NO:27, SEQ ID NO:28, SEQ ID NO:29, or SEQ ID NO:30, including any combination of these forward and reverse primers; (II) a nucleic acid forward primer comprising SEQ ID NO:46, SEQ ID NO:47, or SEQ ID NO:48, and a nucleic acid reverse primer comprising SEQ ID NO:55; SEQ ID NO:56, or SEQ ID NO:57, including any combination of these forward and reverse primers. In certain examples, the method of producing a Brassica plant includes screening each progeny plant sample for sequence (e.g., using a nucleic acid probe) comprising SEQ ID NO:4. Optionally, each sample is screened using a probe comprising SEQ ID NO:4 to detect a PCR amplification product that is amplified using a forward primer comprising SEQ ID NO:16 and a reverse primer comprising SEQ ID NO:28. This method can further include determining zygosity by screening each sample for the wild type N15 chromosome segment, e.g., detecting the presence of sequence (or using a nucleic acid probe) comprising SEQ ID NO:7, and counter-selecting progeny plants that are homozygous for wild type N15 chromosome segment comprising SEQ ID NO:7. Optionally, a probe comprising SEQ ID NO:7 can be used to detect a PCR amplification product that is amplified using a forward primer comprising SEQ ID NO:19 and a reverse primer comprising SEQ ID NO:31.

The foregoing method can be used for introgression of the N15 deletion QTL disclosed herein. After crossing the first Brassica plant with the second Brassica plant, producing and selecting a progeny plant or germplasm thereof having the N15 deletion QTL as described herein, the selected progeny plant is backcrossed with the second plant (the recurrent parent) lacking the N15 deletion QTL to produce a second generation of progeny plants. A plurality of second-generation progeny plants can be screened and at least one second generation progeny plant having the screened-for N15 deletion QTL can be selected in accordance with any method disclosed herein, thereby producing a second-generation progeny plant that combines desirable attributes the recurrent parent with the N15 deletion QTL and its associated low fiber trait disclosed herein. This process can be repeated two, three, four, five, six, or seven times, i.e., by crossing the latest generation of selected backcross progeny plants having the N15 deletion QTL with the recurrent parent plant, and each time identifying and selecting additional backcross progeny plants having N15 deletion QTL. Repeated backcrossing to the recurrent parent plant can be used to create Brassica plant lines that combine (i) the N15 deletion QTL and (ii) the agronomic characteristics of the recurrent parent plant, when backcross lines and recurrent parent are grown in the same environmental conditions.

Further provided is the use of gene editing technology to create a targeted genomic modification of the N15 chromosome in a Brassica genomic locus that produces an N15 deletion disclosed herein. The modification can be done in a Brassica plant, cell, or germplasm that comprises wild-type N15 chromosome (i.e., without the N15 QTL disclosed herein). The modification can include creating a deleted segment located between genomic sequences SEQ ID NO:65 (N101HTV-001) and SEQ ID NO:78 (N101HGY-001). For example, the deletion can be include deleted genomic DNA between position 86.09 cM and position 101.53 cM of chromosome N15, e.g., between position 97.93 cM and position 101.24 cM of chromosome N15. The deleted segment can be at least about 500 kb in length, at least about 532 kb in length, at least about 575 kb in length, at least about 600 kb in length, at least about 700 kb in length, at least about 800 kb in length, at least about 900 kb in length, at least about 1,000 kb in length, or at least about 1,667 kb in length. In some examples, the method can also include inserting a heterologous insertion sequence, i.e., an insertion that has replaced the deleted genomic segment on chromosome N15. Methods for creating such gene edited plants dropouts comprise inducing a first and second double strand break in genomic DNA using a TALE-nuclease (TALEN), a meganuclease, a zinc finger nuclease, or a CRISPR-associated nuclease. In a preferred aspect, the method comprises introducing a CRISPR-associated nuclease and guide RNAs into a B. napus plant cell.

Also provided is a method of making enhanced canola meal or (ECM) that includes identifying, selecting, producing, or using targeted genomic modification to generate a Brassica napus plant comprising a chromosome N15 deletion in accordance with the methods disclosed herein, and crushing the seed from such a plant to generate enhanced canola meal.

The foregoing and other features will become more apparent from the following detailed description of several embodiments.

### BRIEF DESCRIPTION OF THE DRAWINGS

FIG. 1 is a schema showing an alignment of wild type N15 chromosome from canola line NS1822BC with two canola lines (NS7627MC and NS8920BC) containing the N15 deletion QTL disclosed herein; the relative size and location of each N15 deletion shown by the break (absence of) solid line, as indicated by the presence or absence of marker sequences detected in the wild-type line by Illumina XT genotyping assay described herein. All of the markers shown as squares in FIG. 1 are found (produce amplicons) in the wild-type line. Among those markers squares labeled “1” indicate marker sequences detected in NS7627MC but not NS8920BC; squares labeled “2” indicate marker sequences that were not detected in either NS7627MC or NS8920BC; and the square labeled “3” indicates marker sequence detected in NS8920BC but not NS7627MC.

FIG. 2 is schema showing relative locations of forward primers, reverse primers and probes that can be used to assay the presence, absence, and/or zygosity of the N15 deletion. Top line represents wild-type N15 chromosome, bottom line represents N15 with the deletion discovered in NS7627MC, dashed line represents replacement sequence. Arrows refer to primers that can be used to distinguish wild-type and NS7627MC sequences as follows: “1” and “2” indicate forward and revers primers, respectively that amplify wild-type sequence N15 sequence (no deletion); “4 and “5” indicate forward and revers primers, respectively that amplify insertion sequence that has replaced and thereby indicates the presence of N15 deletion. Short lines “3” and “6” indicate probes to detect amplicons produced in wild-type line and N15 deletion line, respectively.

### SEQUENCE LISTING

The nucleic acid sequences listed in the accompanying sequence listing are shown using standard letter abbreviations for nucleotide bases, as defined in 37 C.F.R. § 1.822. Although only one strand of each nucleic acid sequence is shown or referenced, the complementary strand is understood to be included by any reference to the displayed strand.

### DETAILED DESCRIPTION

### I. Terms

Allotetraploid: As used herein, “allotetraploid” generally refers to a hybrid organism that has a chromosome set that is four times that of a haploid organism.

An “allele” is one of several alternative forms of a gene occupying a given locus on a chromosome. When all the alleles present at a given locus on a chromosome are the same, that plant is “homozygous” at that locus. If the alleles present at a given locus on a chromosome differ, that plant is “heterozygous” at that locus.

“Backcrossing” refers to the process whereby hybrid progeny plants are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. Backcrossing has been widely used to introduce new traits into plants. See e.g., Jensen, N., Ed. Plant Breeding Methodology, John Wiley & Sons, Inc., 1988. In a typical backcross protocol, the original variety of interest (recurrent parent) is crossed to a second variety (non-recurrent parent) that carries a gene of interest to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred gene from the nonrecurrent parent.

“Brassica” refers to anyone of Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16).

As used herein, the terms “canola” and “oilseed rape” reference and encompass spring-planted or winter-planted varieties of Brassica napus.

A “Cas protein” refers to a polypeptide encoded by a Cas (CRISPR-associated) gene. A Cas protein includes but is not limited to: a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence.

A “Cas endonuclease” may comprise domains that enable it to function as a double-strand-break-inducing agent. A “Cas endonuclease” may also comprise one or more modifications or mutations that abolish or reduce its ability to cleave a double-strand polynucleotide (dCas). In some aspects, the Cas endonuclease molecule may retain the ability to nick a single-strand polynucleotide (for example, a D10A mutation in a Cas9 endonuclease molecule) (nCas9). When complexed with a guide polynucleotide, the guide polynucleotide/Cas endonuclease complex”, (or “guide polynucleotide/Cas endonuclease system”, “guide polynucleotide/Cas complex”, “guide polynucleotide/Cas system” and “guided Cas system” or “Polynucleotide-guided endonuclease”, “PGEN” ” are capable of directing the Cas endonuclease to a DNA target site, enabling the Cas endonuclease to recognize, bind to, and nick or cleave (introduce a single or double-strand break) the DNA target site. A guided Cas system referred to herein can comprise Cas protein(s) and suitable polynucleotide component(s) of any known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

Commercially useful: As used herein, the term “commercially useful” refers to plant lines and hybrids that have sufficient plant vigor and fertility, such that a crop of the plant line or hybrid can be produced by farmers using conventional farming equipment. In particular embodiments, plant commodity products with described components and/or qualities may be extracted from plants or plant materials of the commercially useful variety. For example, oil comprising desired oil components may be extracted from the seed of a commercially useful plant line or hybrid utilizing conventional crushing and extraction equipment. In another example, enhanced canola meal (defined herein) may be prepared from the crushed seed of commercially useful plant lines which are provided herein and which have a chromosome N15 deletion disclosed herein. In certain embodiments, a commercially useful plant line is an inbred line or a hybrid line. “Agronomically elite” lines and hybrids typically have desirable agronomic characteristics; for example and without limitation: improved yield of at least one plant commodity product; maturity; disease resistance; and standability.

The term “cross” (or “crossed”) refers to the fusion of gametes via pollination to produce progeny (e.g., cells, seeds, and plants). This term encompasses both sexual crosses (i.e., the pollination of one plant by another) and selfing (i.e., self-pollination, for example, using pollen and ovule from the same plant).

The terms “dropout”, “gene dropout”, “knockout” and “gene knockout” refer to a DNA sequence of a cell (e.g. N15 deletion segment) that has been excised from the genome by targeted deletion mediated by a Cas protein or other gene editing tools.

The term “elite line” means any line that has resulted from breeding and selection for superior agronomic performance. An elite plant is any plant from an elite line.

Enhanced canola meal: As used herein, the term “enhanced canola meal” refers to a meal, which is made by processing Brassica napus seeds and which has decreased fiber content, and may have increased protein and true metabolizable energy content, as well as reduced anti-nutritional factors such as glucosinolates, tannins, phytic acid, sinapine and erucic acid. Meal with some or all of these characteristics could allow increasing inclusion rates in the diet of animal species especially in monogastric animals. The enhanced canola meal made from seeds of the N15 deletion Brassica napus oilseed disclosed herein may variously be referred to herein as “ECM” and includes “black seeded canola ECM,” “BSC ECM,” or “dark seeded canola ECM;” the present disclosure is not limited to black-seeded canola and black seeded canola ECM.

Fiber is a component of plant cell walls, and includes carbohydrate polymers (e.g., cellulose (linear glucose polymeric chains)); hemicellulose (branched chains of heteropolymers of, for example, galactose, xylose, arabinose, rhamnose, with phenolic molecules attached); and pectins (water soluble polymers of galacturonic acid, xylose, arabinose, with different degrees of methylation). Fiber also includes polyphenolic polymers (e.g., lignin-like polymers and condensed tannins).

The quality of meal is measured by the percentages of Acid Detergent Fiber (ADF) and Neutral Detergent Fiber (NDF) they contain. The levels of ADF and NDF are critical because they impact animal productivity and digestion. ADF is a measure of the plant components in forages that is least digestible by livestock, including cellulose and lignin. NDF measures most of the structural components in plant cells (i.e. lignin, hemicellulose and cellulose), but not pectin. Decreased ADF and NDF also results in more digestible, higher energy meal.

In particular examples, a seed of a canola plant comprising a germplasm described herein can have a decreased ADF, as compared to a reference canola variety. In certain examples, “high” or “low” component content refers to a comparison between a seed produced by a reference plant comprising a germplasm described herein and a seed produced by standard canola varieties. Thus, a plant having the N15 deletion disclosed herein can produce oilseed with “low” fiber content relative to that of oilseed produced by standard canola varieties and/or an isogenic, parent, or sibling plant that does not have the N15 deletion.

The term “gene” (or “genetic element”) may refer to a heritable genomic DNA sequence with functional significance. A gene includes a nucleic acid fragment that expresses a functional molecule such as, but not limited to, a specific protein, including regulatory sequences preceding (5′ non-coding sequences) and following (3′ non-coding sequences) the coding sequence, as well as intervening intron sequences. The term “gene” may also be used to refer to, for example and without limitation, a cDNA and/or an mRNA encoded by a heritable genomic DNA sequence.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

A “genomic sequence” or “genomic region” is a segment of a chromosome in the genome of a cell that is present on either side of the target site or, alternatively, also comprises the target site or a portion thereof. An “endogenous genomic sequence” refers to genomic sequence within a plant cell.

A “genomic locus” as used herein refers to the genetic or physical location on a chromosome of a gene. As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

The term “genotype” refers to the physical components, i.e., the actual nucleic acid sequence at one or more loci in an individual plant. As used herein, the term “genotype” also refers to the genetic constitution of an individual (or group of individuals) at one or more particular loci. The genotype of an individual or group of individuals is defined and described by the allele forms at the one or more loci that the individual has inherited from its parents. The term genotype may also be used to refer to an individual's genetic constitution at a single locus, at multiple loci, or at all the loci in its genome. A “haplotype” is the genotype of an individual at a plurality of genetic loci. In some examples, the genetic loci described by a haplotype may be physically and genetically linked; i.e., the loci may be positioned on the same chromosome segment.

The term “germplasm” refers to genetic material of or from an individual plant or group of plants (e.g., a plant line, variety, and family), and a clone derived from a plant or group of plants. A germplasm may be part of an organism or cell, or it may be separate (e.g., isolated) from the organism or cell. In general, germplasm provides genetic material with a specific molecular makeup that is the basis for hereditary qualities of the plant. As used herein, “germplasm” refers to cells of a specific plant; seed; tissue of the specific plant (e.g., tissue from which new plants may be grown); and non-seed parts of the specific plant (e.g., leaf, stem, pollen, and cells).

The term “germplasm” is synonymous with “genetic material,” and it may be used to refer to seed (or other plant material) from which a plant may be propagated. In some examples disclosed herein, a germplasm utilized in a method or plant as described herein is from a Brassica line or variety. In particular examples, a germplasm is seed of the Brassica line or variety. In particular examples, a germplasm is a nucleic acid sample from the Brassica line or variety.

The term “introgression” refers to the transmission of an allele at a genetic locus into a genetic background. In some examples, introgression of a specific allele form at the locus may occur by transmitting the allele form to at least one progeny via a sexual cross between two parents of the same species, where at least one of the parents has the specific allele form in its genome. Progeny comprising the specific allele form may be repeatedly backcrossed to a line having a desired genetic background. Backcross progeny may be selected for the specific allele form, so as to produce a new variety wherein the specific allele form has been fixed in the genetic background. In some examples, introgression of a specific allele form may occur by recombination between two donor genomes (e.g., in a fused protoplast), where at least one of the donor genomes has the specific allele form in its genome. Introgression may involve transmission of a specific allele form that may be, for example and without limitation, a selected allele form of a marker allele; a QTL; and/or a transgene. In this disclosure, introgression may involve transmission of a disclosed chromosome N15 deletion into a progeny plant.

An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component. For example and without limitation, a nucleic acid may be isolated from a chromosome by breaking chemical bonds connecting the nucleic acid to the remaining DNA in the chromosome and/or the other material previously associated with the nucleic acid in its cellular milieu (e.g., the nucleus). Nucleic acid molecules and proteins that have been “isolated” include nucleic acid molecules and proteins that are enriched or purified. The term also embraces nucleic acids and proteins prepared by recombinant expression in a host cell, as well as chemically synthesized nucleic acid molecules, proteins, and peptides.

Linkage (dis)equilibrium: As used herein, the term “linkage equilibrium” refers to the situation where two markers independently segregate; i.e., the markers sort randomly among progeny. Markers that show linkage equilibrium are considered unlinked (whether or not they lie on the same chromosome). As used herein, the term “linkage disequilibrium” refers to the situation where two markers segregate in a non-random manner; i.e., the markers have a recombination frequency of less than 50% (and thus by definition, are separated by less than 50 cM on the same linkage group). In some examples, markers that show linkage disequilibrium are considered linked.

Linked, tightly linked, and extremely tightly linked: As used herein, linkage between genes or markers may refer to the phenomenon in which genes or markers on a chromosome show a measurable probability of being passed on together to individuals in the next generation. Thus, linkage of one marker to another marker or gene may be measured and/or expressed as a recombination frequency. The closer two genes or markers are to each other, the closer to “1” this probability becomes. Thus, the term “linked” may refer to one or more genes or markers that are passed together with a gene with a probability greater than 0.5 (which is expected from independent assortment where markers/genes are located on different chromosomes). When the presence of a gene contributes to a phenotype in an individual, markers that are linked to the gene may be said to be linked to the phenotype. Thus, the term “linked” may refer to a relationship between a marker and a gene, or between a marker and a phenotype.

A relative genetic distance (determined by crossing over frequencies and measured in centimorgans (cM)) is generally proportional to the physical distance (measured in base pairs) that two linked markers or genes are separated from each other on a chromosome. One centimorgan is defined as the distance between two genetic markers that show a 1% recombination frequency (i.e., a crossing-over event occurs between the two markers once in every 100 cell divisions). In general, the closer one marker is to another marker or gene (whether the distance between them is measured in terms of genetic distance or physical distance), the more tightly they are linked. Because chromosomal distance is approximately proportional to the frequency of recombination events between traits, there is an approximate physical distance that correlates with recombination frequency. As used herein, the term “linked” may refer to one or more genes or markers that are separated by a genetic distance of less than about 50 cM. Thus, two “linked” genes or markers may be separated by less than about 45 cM; less than about 40 cM; less than about 35 cM; less than about 30 cM; less than about 25 cM; less than about 20 cM; less than about 15 cM; less than about 10 cM; and less than about 5 cM.

As used herein, the term “tightly linked” may refer to one or more genes or markers that are located within about 35 cM of one another. Thus, two “tightly linked” genes or markers may be separated by less than 36 cM; less than 35 cM; less than 34 cM; less than about 33 cM; less than about 32 cM; less than about 31 cM; less than about 30 cM; less than about 29 cM; less than about 28 cM; less than about 27 cM; less than about 26 cM; less than about 25 cM; less than about 24 cM; less than about 23 cM; less than about 22 cM; less than about 21 cM; less than about 20 cM; less than about 19 cM; less than about 18 cM; less than about 17 cM; less than about 16 cM; less than about 15 cM; less than about 14 cM; less than about 13 cM; less than about 12 cM; less than about 11 cM; less than about 10 cM; less than about 9 cM; less than about 8 cM; less than about 7 cM; less than about 6 cM; less than about 5 cM; and even smaller genetic distances.

As used herein, the term “extremely tightly-linked” may refer to one or more genes or markers that are located within about 5.0 cM of one another. Thus, two “extremely tightly-linked” genes or markers may be separated by less than 6.0 cM; less than 5.5 cM; less than 5.0 cM; less than about 4.5 cM; less than about 4.0 cM; less than about 3.5 cM; less than about 3.0 cM; less than about 2.5 cM; less than about 2.0 cM; less than about 1.5 cM; less than about 1.0 cM; and less than about 0.5 cM.

The closer a particular marker is to a gene that encodes a polypeptide that contributes to a particular phenotype (whether measured in terms of genetic or physical distance), the more tightly linked is the particular marker to the phenotype. In view of the foregoing, it will be appreciated that markers linked to a particular gene or phenotype include those markers that are tightly linked, and those markers that are extremely tightly linked, to the gene or phenotype. In some embodiments, the closer a particular marker is to a gene that contributes to low fiber content phenotype (whether measured in terms of genetic or physical distance), the more tightly linked is the particular marker to the low fiber content phenotype. Thus, linked, tightly linked, and extremely tightly linked genetic markers of a low fiber content phenotype in Brassica may be useful in MAS programs to identity Brassica varieties comprising low fiber content (when compared to parental varieties and/or at least one particular conventional variety), to identify individual Brassica plants comprising low fiber content, and to breed this trait into other Brassica varieties (e.g., “AC” genome, such as B. napus) to decrease fiber content.

Marker: Unlike DNA sequences that encode proteins, which are generally well-conserved within a species, other regions of DNA (e.g., non-coding DNA and introns) tend to develop and accumulate polymorphism, and therefore may be variable between individuals of the same species. The genomic variability can be of any origin, for example, the variability may be due to DNA insertions, deletions, duplications, repetitive DNA elements, point mutations, recombination events, and the presence and sequence of transposable elements. Such regions may contain useful molecular genetic markers. In general, any differentially inherited polymorphic trait (including nucleic acid polymorphisms) that segregates among progeny is a potential marker.

As used herein, the terms “marker” and “molecular marker” refer to a nucleic acid or encoded product thereof (e.g., a protein) used as a point of reference when identifying a linked locus. Thus, a marker may refer to a gene or nucleic acid that can be used to identify plants having a particular allele. A marker may be described as a variation at a given genomic locus. A genetic marker may be a short DNA sequence, such as a sequence surrounding a single base-pair change (single nucleotide polymorphism, or “SNP”), or a long one, for example, a microsatellite/simple sequence repeat (“SSR”). A “marker allele” or “marker allele form” refers to the version of the marker that is present in a particular individual. The term “marker” as used herein may refer to a cloned segment of chromosomal DNA and may also or alternatively refer to a DNA molecule that is complementary to a cloned segment of chromosomal DNA. The term also refers to nucleic acid sequences complementary to genomic marker sequences, such as nucleic acid primers and probes.

A marker may be described, for example, as a specific polymorphic genetic element at a specific location in the genetic map of an organism. A genetic map may be a graphical representation of a genome (or a portion of a genome, such as a single chromosome) where the distances between landmarks on the chromosome are measured by the recombination frequencies between the landmarks. A genetic landmark can be any of a variety of known polymorphic markers, for example and without limitation: simple sequence repeat (SSR) markers; restriction fragment length polymorphism (RFLP) markers; and single nucleotide polymorphism (SNP) markers. As one example, SSR markers can be derived from genomic or expressed nucleic acids (e.g., expressed sequence tags (ESTs)).

Additional markers include, for example and without limitation, ESTs; amplified fragment length polymorphisms (AFLPs) (Vos et al., 1995, Nucl. Acids Res. 23:4407; Becker et al., 1995, Mol. Gen. Genet. 249:65; Meksem et al., 1995, Mol. Gen. Genet. 249:74); randomly amplified polymorphic DNA (RAPD); and isozyme markers. Isozyme markers may be employed as genetic markers, for example, to track isozyme markers or other types of markers that are linked to a particular first marker. Isozymes are multiple forms of enzymes that differ from one another with respect to amino acid sequence (and therefore with respect to their encoding nucleic acid sequences). Some isozymes are multimeric enzymes containing slightly different subunits. Other isozymes are either multimeric or monomeric, but have been cleaved from a pro-enzyme at different sites in the pro-enzyme amino acid sequence. Isozymes may be characterized and analyzed at the protein level or at the nucleic acid level. Thus, any of the nucleic acid-based methods described herein can be used to analyze isozyme markers in particular examples.

Accordingly, genetic marker alleles that are polymorphic in a population can be detected and distinguished by one or more analytic methods such as, PCR-based sequence specific amplification methods, RFLP analysis, AFLP analysis, isozyme marker analysis, SNP analysis, SSR analysis, allele specific hybridization (ASH) analysis, detection of amplified variable sequences of the plant genome, detection of self-sustained sequence replication, detection of simple sequence repeats (SSRs), randomly amplified polymorphic DNA (RAPD) analysis. Thus, in certain examples disclosed herein, such known methods can be used to detect the Variant 2 allele as well as markers for detecting the presence or absence of the Variant 2 allele disclosed herein.

Numerous statistical methods for determining whether markers are genetically linked to a QTL (or to another marker) are known to those of skill in the art and include, for example and without limitation, standard linear models (e.g., ANOVA or regression mapping; Haley and Knott, 1992, Heredity 69:315); and maximum likelihood methods (e.g., expectation-maximization algorithms; Lander and Botstein, 1989, Genetics 121:185-99; Jansen, 1992, Theor. Appl. Genet. 85:252-60; Jansen, 1993, Biometrics 49:227-31; Jansen, 1994, “Mapping of quantitative trait loci by using genetic markers: an overview of biometrical models,” In J. W. van Ooijen and J. Jansen (eds.), Biometrics in Plant breeding: applications of molecular markers, pp. 116-24 (CPRO-DLO Netherlands); Jansen, 1996, Genetics 142:305-11; and Jansen and Stam, 1994, Genetics 136:1447-55).

Exemplary statistical methods include single point marker analysis; interval mapping (Lander and Botstein, 1989, Genetics 121:185); composite interval mapping; penalized regression analysis; complex pedigree analysis; MCMC analysis; MQM analysis (Jansen, 1994, Genetics 138:871); HAPLO-IM+ analysis, HAPLO-MQM analysis, and HAPLO-MQM+ analysis; Bayesian MCMC; ridge regression; identity-by-descent analysis; and Haseman-Elston regression, any of which are suitable in the context of particular embodiments of the invention. Alternative statistical methods applicable to complex breeding populations that may be used to identify and localize QTLs in particular examples are described in U.S. Pat. No. 6,399,855 and PCT International Patent Publication No. WO0149104 A2. All of these approaches are computationally intensive and are usually performed with the assistance of a computer-based system comprising specialized software. Appropriate statistical packages are available from a variety of public and commercial sources, and are known to those of skill in the art.

“Marker-assisted selection” (MAS) is a process by which phenotypes are selected based on marker genotypes. Marker assisted selection includes the use of marker genotypes for identifying plants for inclusion in and/or removal from a breeding program or planting.

Molecular marker technologies generally increase the efficiency of plant breeding through MAS. A molecular marker allele that demonstrates linkage disequilibrium with a desired phenotypic trait (e.g., a QTL) provides a useful tool for the selection of the desired trait in a plant population. The key components to the implementation of an MAS approach are the creation of a dense (information rich) genetic map of molecular markers in the plant germplasm; the detection of at least one QTL based on statistical associations between marker and phenotypic variability; the definition of a set of particular useful marker alleles based on the results of the QTL analysis; and the use and/or extrapolation of this information to the current set of breeding germplasm to enable marker-based selection decisions to be made.

The closer a particular marker is to a gene that encodes a polypeptide that contributes to a particular phenotype (whether measured in terms of genetic or physical distance), the more tightly linked is the particular marker to the phenotype. In view of the foregoing, it will be appreciated that the closer (whether measured in terms of genetic or physical distance) that a marker is linked to a particular gene, the more likely the marker is to segregate with that gene (e.g., the N15 deletion disclosed herein) and its associated phenotype (e.g., the contribution to reduced fiber of the N15 deletion disclosed herein). Thus, the genetic markers disclosed herein can be used in MAS programs to identity Brassica varieties that have or can generate progeny that have reduced fiber (when compared to parental varieties, siblings and/or otherwise isogenic plants lacking the N15 deletion), to identify individual Brassica plants comprising this reduced fiber QTL, and to breed this QTL into other Brassica varieties to reduce their fiber content.

A “marker set” or a “set” of markers or probes refers to a specific collection of markers (or data derived therefrom) that may be used to identify individuals comprising a trait of interest. In some examples, a set of markers linked to N15 deletion may be used to identify a Brassica plant comprising one or more copies of an N15 deletion disclosed herein. Data corresponding to a marker set (or data derived from the use of such markers) may be stored in an electronic medium. While each marker in a marker set may possess utility with respect to trait identification, individual markers selected from the set and subsets including some, but not all, of the markers may also be effective in identifying individuals comprising the trait of interest.

As used herein, the term “mapping population” may refer to a plant population (e.g., a Brassica plant population) used for genetic mapping. Mapping populations are typically obtained from controlled crosses of parent genotypes, as may be provided by two inbred lines. Decisions on the selection of parents, mating design for the development of a mapping population, and the type of markers used depend upon the gene to be mapped, the availability of markers, and the molecular map. The parents of plants within a mapping population should have sufficient variation for a trait(s) of interest at both the nucleic acid sequence and phenotype level. Variation of the parents' nucleic acid sequence is used to trace recombination events in the plants of the mapping population.

The availability of informative polymorphic markers is dependent upon the amount of nucleic acid sequence variation. Thus, a particular informative marker may not be identified in a particular cross of parent genotypes, though such markers may exist.

A “genetic map” is a description of genetic linkage relationships among loci on one or more chromosomes (or linkage groups) within a given species, as may be determined by analysis of a mapping population. In some examples, a genetic map may be depicted in a diagrammatic or tabular form. The term “genetic mapping” may refer to the process of defining the linkage relationships of loci through the use of genetic markers, mapping populations segregating for the markers, and standard genetic principles of recombination frequency. A “genetic map location” refers to a location on a genetic map (relative to surrounding genetic markers on the same linkage group or chromosome) where a particular marker can be found within a given species. In contrast, a “physical map of the genome” refers to absolute distances (for example, measured in base pairs or isolated and overlapping contiguous genetic fragments) between markers within a given species. A physical map of the genome does not necessarily reflect the actual recombination frequencies observed in a test cross of a species between different points on the physical map.

As used herein, a “mutated gene” or “modified gene” is a gene that has been altered through human intervention. Such a “mutated” or “modified” gene has a sequence that differs from the sequence of the corresponding non-mutated gene by at least one nucleotide addition, deletion, or substitution. In certain embodiments of this disclosure, the mutated or modified gene comprises an excision or deletion of a sequence of nucleotides within that results from two double strands break which are specifically targeted within a genomic sequence by guide polynucleotide/Cas endonuclease system or a gene edited tool as disclosed herein. A “gene edited” or “modified” plant is a plant comprising a mutated gene or deletion. As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

As used herein the term “native gene” refers to a gene as found in its natural endogenous location with its own regulatory sequences. In the context of this disclosure, a “mutated” or “modified” gene is not a native gene.

As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” The term includes single- and double-stranded forms of DNA. A nucleic acid molecule can include either or both naturally occurring and modified nucleotides, linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, inter-nucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Quantitative trait: As used herein, a “quantitative trait” may refer to a trait or phenotype that is expressed in varying degrees, along a generally continuous gradient and is frequently linked to two or more genes and is affected by environment.

Quantitative trait locus or QTL: As used herein, a “quantitative trait locus” refers to a segment or region of DNA containing or genetically linked to a gene or genomic region underlying a quantitative trait.

As used herein, the term “QTL interval” may refer to stretches of DNA that are linked to the gene(s) that underlie the QTL trait. A QTL interval is typically, but not necessarily, larger than the QTL itself. A QTL interval may contain stretches of DNA that are 5′ and/or 3′ with respect to the QTL.

Multiple experimental paradigms have been developed to identify and analyze QTLs. See, e.g., Jansen (1996) Trends Plant Sci. 1:89. The majority of published reports on QTL mapping in crop species have been based on the use of a bi-parental cross. See Lynch and Walsh (1997) Genetics and Analysis of Quantitative Traits, Sinauer Associates, Sunderland. Typically, these paradigms involve crossing one or more parental pairs that can be, for example, a single pair derived from two inbred strains, or multiple related or unrelated parents of different inbred strains or lines, which each exhibit different characteristics relative to the phenotypic trait of interest. Typically, this experimental protocol involves deriving 100 to 300 segregating progeny from a single cross of two divergent inbred lines that are, for example, selected to maximize phenotypic and molecular marker differences between the lines. The parents and segregating progeny are genotyped for multiple marker loci, and evaluated for one to several quantitative traits (e.g., low fiber content). QTLs are then identified as significant statistical associations between genotypic values and phenotypic variability among the segregating progeny.

Numerous statistical methods for determining whether markers are genetically linked to a QTL (or to another marker) are known to those of skill in the art and include, for example and without limitation, standard linear models (e.g., ANOVA or regression mapping; Haley and Knott (1992) Heredity 69:315); and maximum likelihood methods (e.g., expectation-maximization algorithms; Lander and Botstein (1989) Genetics 121:185-99; Jansen (1992) Theor. Appl. Genet. 85:252-60; Jansen (1993) Biometrics 49:227-31; Jansen (1994) “Mapping of quantitative trait loci by using genetic markers: an overview of biometrical models,” In J. W. van Ooijen and J. Jansen (eds.), Biometrics in Plant breeding: applications of molecular markers, pp. 116-24, CPRO-DLO Netherlands; Jansen (1996) Genetics 142:305-11; and Jansen and Stam (1994) Genetics 136:1447-55).

Exemplary statistical methods include single point marker analysis; interval mapping (Lander and Botstein (1989) Genetics 121:185); composite interval mapping; penalized regression analysis; complex pedigree analysis; MCMC analysis; MQM analysis (Jansen (1994) Genetics 138:871); HAPLO-IM+ analysis, HAPLO-MQM analysis, and HAPLO-MQM+ analysis; Bayesian MCMC; ridge regression; identity-by-descent analysis; and Haseman-Elston regression, any of which are suitable in the context of particular embodiments disclosed herein. Alternative statistical methods applicable to complex breeding populations that may be used to identify and localize QTLs in particular examples are described in U.S. Pat. No. 6,399,855 and PCT International Patent Publication No. WO0149104 A2. All of these approaches are computationally intensive and are usually performed with the assistance of a computer-based system comprising specialized software. Appropriate statistical packages are available from a variety of public and commercial sources, and are known to those of skill in the art.

As used herein, the term “single-nucleotide polymorphism” (SNP) may refer to a DNA sequence variation occurring when a single nucleotide in the genome (or other shared sequence) differs between members of a species or paired chromosomes in an individual. In some examples, markers linked to low fiber content are SNP markers. Recent high-throughput genotyping technologies such as GoldenGate® and INFINIUM® assays (Illumina, San Diego, CA) may be used in accurate and quick genotyping methods by multiplexing SNPs from 384-plex to >100,000-plex assays per sample. Other exemplary technologies for interrogating SNPs include nucleic acid sequencing (e.g., next-generation sequencing or NGS), primer extension, allele-specific PCR (e.g. KASP), H2-dependent PCR (rhPCR), Melt Analysis of Mismatch Amplification Mutation Assay (Melt-MAMA), Masscode™ (Qiagen, Germantown, Md.), Invader® (Hologic, Madison, Wis.), Serial Invasive Signal Amplification Reaction (SISAR), SnapShot® (Applied Biosystems, Foster City, Calif.), and Taqman® (Applied Biosystems, Foster City, Calif.). Although SNP markers are highly useful, availability of high-quality DNA sequence information is necessary for their discovery.

Plant: As used herein, the term “plant” may refer to a whole plant, a cell or tissue culture derived from a plant, and/or any part of any of the foregoing. Thus, the term “plant” encompasses, for example and without limitation, whole plants; plant components and/or organs (e.g., leaves, stems, and roots); plant tissue; seed; and a plant cell. A plant cell may be, for example and without limitation, a cell in and/or of a plant, a cell isolated from a plant, and a cell obtained through culturing of a cell isolated from a plant. Thus, the term “Brassica plant” may refer to, for example and without limitation, a whole plant; multiple plants; Brassica plant cell(s); plant protoplast; plant tissue culture (e.g., from which a whole plant can be regenerated); plant callus; plant parts (e.g., seed, flower, cotyledon, leaf, stem, bud, root, and root tip); and plant cells that are intact in a plant or in a part of a plant.

Plant line: As used herein, a “line” refers to a group of plants that display little genetic variation (e.g., no genetic variation) between individuals for at least one trait. Inbred lines may be created by several generations of self-pollination and selection or, alternatively, by vegetative propagation from a single parent using tissue or cell culture techniques. As used herein, the terms “cultivar,” “variety,” and “type” are synonymous, and these terms refer to a line that is used for commercial production.

A “variety” or “cultivar” is a plant line that is used for commercial production which is distinct, stable and uniform in its characteristics when propagated. In the case of a hybrid variety or cultivar, the parental lines are distinct, stable, and uniform in their characteristics.

Plant commodity product: As used herein, the term “plant commodity product” refers to commodities produced from a particular plant or plant part (e.g., a plant comprising germplasm disclosed herein, and a plant part obtained from a plant comprising germplasm disclosed herein). A commodity product may be, for example and without limitation: grain; meal; forage; protein; isolated protein; flour; oil; crushed oilseed (as prepared prior to processing into separate oil and meal products), or whole grains/oilseeds; any food product comprising any meal, oil; or silage.

Trait or phenotype: The terms “trait” and “phenotype” are used interchangeably herein. For the purposes of the present disclosure, the traits of particular interest are low fiber content and, in some cases, seed coat color. Some canola varieties exhibit a yellow seed coat, while further varieties exhibit a dark (e.g., black, dark, and mottled) seed coat.

Seed color: Canola varieties (e.g., inbred canola lines and hybrids) can be characterized by seed color. Canola seed color rating or “seed color” is generally scored on a 1-5 scale, based on seeds obtained from healthy plants at or near complete seed maturity. “1” signifies a good yellow color. “2” signifies mainly yellow with some brown. “3” indicates a mixture of brown and yellow. “4” and “5” signify brown and black, respectively.

### III. Detection of N15 Deletion in Brassica napus

Compositions and methods disclosed herein include the N15 deletion and the associated markers shown herein to be linked to low fiber content. This N15 deletion can be found, for example, in commercially available PROTECTOR lines from Pioneer, such as, P508MCL, PV560GM and HARVESTMAX canola hybrids 45M35 and 45M38. Seeds of these lines have been deposited with the American Type Culture Collection (ATCC), 10801 University Boulevard, Manassas, Va. 20110-2209 USA and given the following accession numbers: for 45M35—ATCC Accession No. PTA-123487 (see U.S. Pat. No. 10,085,403); for 45M38—ATCC Accession No. PTA-124074 (see U.S. Pat. No. 10,440,917); and for PV560GM—ATCC Accession Nos. PTA-123488 and/or PTA-123489 (See U.S. Pat. No. 10,076,094).

Such markers may be used, for example and without limitation, to identify canola plants and germplasm having an increased likelihood of comprising a low fiber content phenotype; to select such canola plants and germplasm (e.g., in a marker-assisted selection program); and to identify and select canola plants and germplasm that do not have an increased likelihood of comprising a low fiber content phenotype. Use of one or more probe sequences and markers describe herein may provide advantages to plant breeders with respect to the time, cost, and labor involved in canola breeding, when compared to currently available compositions and methods in the art. For example, one or more of the probe or marker sequences described herein may provide superior results in marker-assisted breeding of low fiber content in canola, when compared to currently available markers for this purpose.

Methods for detecting (identifying) Brassica napus plants or germplasm that carry particular alleles of low fiber content markers are a feature of some embodiments. In some embodiments, any of a variety of marker detection protocols available in the art may be used to detect a marker allele, depending on the type of marker being detected. In examples, suitable methods for marker detection may include amplification and identification of the resulting amplified marker by, for example and without limitation, PCR; LCR; and transcription-based amplification methods (e.g., SNP detection, SSR detection, RFLP analysis, and many others).

In general, a genetic marker relies on one or more property of nucleic acids for its detection. For example, some techniques for detecting genetic markers utilize hybridization of a probe nucleic acid to a nucleic acid corresponding to the genetic marker (e.g., an amplified nucleic acid produced using a genomic Brassica napus DNA molecule as a template). Hybridization formats including, for example and without limitation, solution phase; solid phase; mixed phase; and in situ hybridization assays may be useful for allele detection in particular embodiments. An extensive guide to the hybridization of nucleic acids may be found, for example, in Tijssen (1993) Laboratory Techniques in Biochemistry and Molecular Biology-Hybridization with Nucleic Acid Probes Elsevier, NY.

Markers corresponding to genetic polymorphisms between members of a population may be detected by any of numerous methods including, for example and without limitation, nucleic acid amplification-based methods; and nucleotide sequencing of a polymorphic marker region. Many detection methods (including amplification-based and sequencing-based methods) may be readily adapted to high throughput analysis in some examples, for example, by using available high throughput sequencing methods, such as sequencing by hybridization.

Accordingly, this disclosure further provides methods of identifying and/or selecting a low fiber content Brassica napus plant or germplasm, comprising: (a) detecting, in said Brassica napus plant or germplasm, the presence of one or more genetic markers associated with low fiber content in a Brassica napus plant, as described herein; and (b) selecting said Brassica napus plant or germplasm based on the presence of the one or more genetic markers associated with low fiber content in a Brassica napus plant.

Additionally, the methods disclosed herein include detecting an amplified DNA fragment associated with the presence of a particular allele of a SNP. In some examples, the amplified fragment associated with a particular allele of a SNP has a predicted nucleic acid sequence, and detecting an amplified DNA fragment having the predicted nucleic acid sequence is performed such that the amplified DNA fragment has the nucleic acid sequence that corresponds (e.g., a homology of at least about 80%, 90%, 95%, 96%, 97%, 98%, 99% or more) to the expected sequence based on the sequence of the marker associated with that SNP in the plant in which the marker was first detected.

The detecting of a particular allele of a SNP can be performed by any of a number or techniques, including, but not limited to, the use of detectable labels. Detectable labels suitable for use include any composition detectable by spectroscopic, radioisotopic, photochemical, biochemical, immunochemical, electrical, optical, or chemical means. Thus, a particular allele of a SNP may be detected using, for example, autoradiography, fluorography, or other similar detection techniques, depending on the particular label to be detected. Useful labels include biotin (for staining with labeled streptavidin conjugate), magnetic beads, fluorescent dyes, radiolabels, enzymes, and colorimetric labels. Other labels include ligands that bind to antibodies or specific binding targets labeled with fluorophores, chemiluminescent agents, and enzymes. In some examples described herein, detection techniques include the use of fluorescent dyes.

Several methods are available for SNP genotyping, including but not limited to, hybridization, primer extension, oligonucleotide ligation, nuclease cleavage, minisequencing and coded spheres. Such methods have been reviewed in various publications: Gut, Hum. Mutat. 17:475 (2001); Shi, Clin. Chem. 47:164 (2001); Kwok, Pharmacogenomics 1:95 (2000); Bhattramakki and Rafalski, Discovery and application of single nucleotide polymorphism markers in plants, in PLANT GENOTYPING: THE DNA FINGERPRINTING OF PLANTS, CABI Publishing, Wallingford (2001). A wide range of commercially available technologies utilize these and other methods to interrogate SNPs, including Masscode™ (Qiagen, Germantown, Md.), Invader® (Hologic, Madison, Wis.), SnapShot® (Applied Biosystems, Foster City, Calif.), Taqman® (Applied Biosystems, Foster City, Calif.) and Infinium Bead Chip™ (Illumina, San Diego, Calif.). In some examples disclosed herein, the method of SNP genotyping includes the use of the Infinium Bead Chip™.

Marker sequences and probes for certain markers of N15 deletion QTL disclosed herein are shown in Table 4 and Table 7 herein. These are associated with low fiber content. One marker or a combination of markers can be used to detect the presence of a low fiber content plant. For example, a marker can be located within the N15 deletion QTL or be present in the genome of the plant as a haplotype as defined herein.

Accordingly, in some aspects of the present disclosure, a method of selecting, detecting and/or identifying a low fiber content Brassica napus plant or germplasm is provided, the method comprising: detecting, in said Brassica napus plant or germplasm, the presence of a marker (e.g., a marker allele) associated with N15 deletion QTL in a Brassica napus plant, wherein said marker is located within a chromosomal interval. The chromosomal interval can comprise, consist essentially of, or consist of a chromosome interval on chromosome N15 defined by and including SEQ ID NO:65 (N101HTV-001) and SEQ ID NO:78 (N101HGY-001) and the N15 deletion is associated with low fiber content in Brassica napus. For example, the disclosed method can include screening the sample for the absence of one or more markers located in genomic DNA between genomic position 97.93 cM and position 101.24 cM of chromosome N15, e.g., between position 86.09 and position 101.53 cM of chromosome N15, thereby identifying and/or selecting a low fiber content Brassica napus plant or germplasm. Also, the chromosomal interval can comprise, consist essentially of, or consist of a chromosome interval on chromosome N15 defined by and including bp position that correspond to physical nucleotide positions from about 51,500,326 to about 53,229,532 in chromosome N15; e.g., from about 52,639,876 to about 53,229,532 or from about 51,500,326 to about 53,185,850—nucleotide positions in publicly available Brasssica napus reference genome for Darmor V10. See Rousseau-Gueutin et al., 2020, Gigascience 9(12) giaa137. 10.1093/gigascience/giaa137.

### V. Introgression of Markers for Low Fiber Content into Brassica

As set forth, supra, identification of Brassica plants or germplasm that includes a marker allele or alleles that is/are linked to a low fiber content phenotype provides a basis for performing marker assisted selection of Brassica. For example, at least one Brassica plant that comprises at least one marker allele for the disclosed N15 deletion QTL is selected. Brassica plants that comprise marker alleles indicating the presence of the N15 deletion segment (i.e., absence of the deletion) may be selected against.

This disclosure thus provides methods for selecting a Brassica plant exhibiting low fiber content comprising detecting in the plant the presence of one or more genetic markers associated with the N15 deletion QTL described herein. This disclosure provides a method for selecting such a plant, the method comprises providing a sample of genomic DNA from a Brassica plant; and using any of the methods disclosed herein for detecting in the sample of genomic DNA at least one genetic marker or sequence indicating the sample comprises the N15 deletion QTL described herein. Detecting can comprise detecting one or more marker, a combination of markers (haplotype), detecting the absence of the N15 deletion segment, and/or the presence of a heterologous insertion sequence, i.e., an insertion that has replaced the deleted genomic segment on chromosome N15.

The disclosure provides a method comprising the transfer by introgression of the nucleic acid sequence from a N15 deletion QTL donor Brassica plant into a standard or higher fiber content recipient Brassica plant by crossing the plants. This transfer can be accomplished by using traditional breeding techniques. The N15 deletion locus associated with low fiber content can be introgressed into elite or commercial Brassica varieties using marker-assisted selection (MAS) or marker-assisted breeding (MAB). MAS and MAB involve the use of one or more of the molecular markers, identified as having a significant likelihood of co-segregation with a desired trait, and used for the identification and selection of those offspring plants that contain one or more of the genes that encode the desired trait. As disclosed herein, such identification and selection are based on the selection of one or more marker sequences located in one of the N15 QTL intervals disclosed herein or one or more markers associated with N15 deletions disclosed herein. MAB can also be used to develop near-isogenic lines (NIL) harboring one or more low fiber content alleles of interest, allowing a more detailed study of an effect of such allele(s), and is also an effective method for development of backcross inbred line (BIL) populations. Brassica plants developed according to these embodiments can, in some examples, derive a majority of their traits from the recipient plant and derive the low fiber content trait from the donor plant. MAB/MAS techniques increase the efficiency of backcrossing and introgressing genes using marker-assisted selection (MAS) or marker-assisted breeding (MAB).

Thus, traditional breeding techniques can be used to introgress a nucleic acid sequence associated with low fiber content into a high fiber content recipient Brassica plant. For example, inbred low fiber content Brassica plant lines can be developed using the techniques of recurrent selection and backcrossing, selfing, and/or dihaploids, or any other technique used to make parental lines. In a method of recurrent selection and backcrossing, low fiber content can be introgressed into a target recipient plant (the recurrent parent) by crossing the recurrent parent with a first donor plant, which differs from the recurrent parent and is referred to herein as the “non-recurrent parent.” The recurrent parent is a plant that has high fiber content and, in some cases, comprises commercially desirable characteristics, such as, but not limited to disease and/or insect resistance, valuable nutritional characteristics, valuable abiotic stress tolerance (including, but not limited to, drought tolerance, salt tolerance), and the like. In some cases, the non-recurrent parent exhibits low fiber content and comprises a nucleic acid sequence that is associated with low fiber content. The non-recurrent parent can be any plant variety or inbred line that is cross-fertile with the recurrent parent.

In certain examples of the disclosed introgression method, the progeny resulting from a cross between the recurrent parent and non-recurrent parent are backcrossed to the recurrent parent. The resulting plant population is then screened for the desired characteristics, which screening can occur in a number of different ways. For instance, the population can be screened using phenotypic pathology screens or quantitative bioassays as are known in the art. Alternatively, instead of using bioassays, MAB can be performed using one or more of the herein before described molecular markers to identify those progeny that comprise a nucleic acid sequence associated with low fiber content. Also, MAB can be used to confirm the results obtained from the quantitative bioassays. In some embodiments, the markers defined herein are suitable to select proper offspring plants by genotypic screening.

Following screening, the F1 hybrid plants that exhibit a low fiber content phenotype or, in some embodiments, the genotype, and thus comprise the requisite nucleic acid sequence associated with low fiber content, can then be selected and backcrossed to the recurrent parent for one or more generations in order to allow for the Brassica plant to become increasingly inbred. This process can be performed for one, two, three, four, five, six, seven, eight, or more generations.

Accordingly, the sequences and markers disclosed herein which are associated with the N15 deletion QTL can be used in MAS methods to identify and/or select and/or produce progeny having the N15 deletion QTL associated with low fiber content. Therefore, the present disclosure provides a method of selecting a low fiber content Brassica plant, the method comprising: detecting, in a Brassica germplasm, the presence of a sequence or marker associated with the N15 deletion QTL a Brassica plant, wherein said sequence or marker is located within the chromosomal interval disclosed herein, and selecting a plant from said germplasm, thereby selecting a plant having the N15 deletion QTL associated with lower fiber content. The disclosed chromosomal interval can be the interval defined by and including SEQ ID NO:65 (N101HTV-001) and SEQ ID NO:78 (N101HGY-001) and the N15 deletion is associated with low fiber content in Brassica napus. For example, the disclosed method can include screening the sample for the absence of one or more genomic markers located between genomic position 86.09 cM and position 101.53 cM of chromosome N15, e.g., between position 97.93 cM and position 101.24 cM of chromosome N15, thereby identifying and/or selecting a low fiber content Brassica napus plant or germplasm. Also, the chromosomal interval can comprise, consist essentially of, or consist of a chromosome interval on chromosome N15 defined by and including bp position that correspond to physical nucleotide positions physical nucleotide positions 52,639,876 to about 53,229,532 or from about physical nucleotide 51,500,326 to about 53,185,850 in publicly available Brassica napus reference genome for Darmor V10. See Rousseau-Gueutin et al., 2020.

Also provided herein is a method of producing a low fiber content plant and/or germplasm, the method comprising: crossing a first canola plant or germplasm with a second canola plant or germplasm, wherein said first canola plant or germplasm comprises within its genome the N15 deletion QTL disclosed herein, collecting seed from the cross and growing a progeny canola plant from the seed, wherein said progeny canola plant comprises in its genome said the N15 deletion QTL associated with low fiber content and can be used to produce a low fiber content canola plant.

In some examples, the second canola plant or germplasm used in the method is of an elite variety of canola. In some examples, the crossing of the first and second canola plants produces a progeny canola plant or germplasm having the low fiber content marker introgressed into a genome that is at least about 50%, 55%, 60%, 65%, 70%, 75%, 80%, 85%, 90%, 95%, 97%, 99% identical to that of an elite variety of canola.

The disclosed method can be used to introgress the N15 deletion QTL associated with low fiber content disclosed herein into a genetic background lacking said deletion QTL, the method comprising: crossing a donor comprising said N15 deletion with a recurrent parent that comprises the N15 deletion segment (i.e., lacks the N15 deletion); and backcrossing progeny comprising N15 deletion with the recurrent parent, wherein said progeny are identified by detecting in their genome the presence of a sequence or marker indicating the presence of the N15 deletion QTL. The method produces a low fiber content canola plant or germplasm comprising said genetic marker associated with low fiber content in the genetic background of the recurrent parent, thereby introgressing the genetic marker associated with low fiber content into a genetic background lacking said marker.

The present disclosure provides Brassica plants and germplasms having low fiber content. As discussed above, the disclosed methods can be utilized to identify, select and/or produce a canola plant or germplasm having the disclosed N15 deletion QTL which is associated with low fiber content. In addition to the methods described above, a Brassica plant or germplasm having low fiber content may be produced by any method whereby an N15 deletion disclosed herein is introduced into the Brassica plant or germplasm by such methods that include, but are not limited to, transformation (including, but not limited to, bacterial-mediated nucleic acid delivery (e.g., via Agrobacteria)), viral-mediated nucleic acid delivery, silicon carbide or nucleic acid whisker-mediated nucleic acid delivery, liposome mediated nucleic acid delivery, microinjection, microparticle bombardment, electroporation, sonication, infiltration, PEG-mediated nucleic acid uptake, as well as any other electrical, chemical, physical (mechanical) and/or biological mechanism that results in the introduction of nucleic acid into the plant cell, or any combination thereof, protoplast transformation or fusion, a double haploid technique, embryo rescue, or by any other nucleic acid transfer system.

“Introducing” in the context of a plant cell, plant and/or plant part means contacting a nucleic acid molecule with the plant, plant part, and/or plant cell in such a manner that the nucleic acid molecule gains access to the interior of the plant cell and/or a cell of the plant and/or plant part. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell.

Thus, a canola plant, or part thereof, having a genetic marker associated with low fiber content, obtainable by the methods of the presently disclosed subject matter, are aspects of the presently disclosed subject matter.

The canola plant or germplasm may be the progeny of a cross between an elite variety of canola and a variety of canola that comprises an allele associated with low fiber content. In some examples, the canola plant or germplasm is at least about 50%, 55%, 60%, 65%, 70%, 75%, 80%, 85%, 90%, 95%, 97%, 99% or 100% identical to that of an elite variety of canola.

The canola plant or germplasm may be the progeny of an introgression wherein the recurrent parent is an elite variety of canola and the donor comprises a genetic marker associated (e.g., SNP, combination of SNPs, SNP located in a chromosome interval) with low fiber content in a canola plant as described herein.

The canola plant or germplasm may be the progeny of a cross between a first elite variety of canola (e.g., a tester line) and the progeny of a cross between a second elite variety of canola (e.g., a recurrent parent) and a variety of canola that comprises a genetic marker associated with low fiber content in a canola plant as described herein (e.g., a donor).

Another aspect of the presently disclosed subject matter relates to a method of producing seeds that can be grown into low fiber content canola plants. In some examples, the method comprises providing a low fiber content canola plant of this invention, crossing the low fiber content canola plant with another canola plant, and collecting seeds resulting from the cross, which when planted, produce low fiber content canola plants.

Accordingly, the provided herein are improved canola plants, seeds, and/or canola tissue culture produced by the methods described herein.

In some examples, the presently disclosed subject matter provides methods for analyzing the genomes of canola plants/germplasms to identify those that include desired markers associated with low fiber content. In some examples, the methods of analysis comprise amplifying subsequences of the genomes of the canola plants/germplasms and determining the nucleotides present in one, some, or all positions of the amplified subsequences.

Thus, the present disclosure provides methods for detecting alleles associated with low fiber content in canola. In some examples, allele discrimination is performed in a microtiter plate using Infinium Bead Chip™ technology and GoldenGate™ allele-specific extension PCR-based assay (Illumina, San Diego, CA), which identifies each SNP with a discrete fluorescent tag and a unique address to target a particular bead in the array. In further examples, the reaction products or fluorescent intensities on the beads are captured and the SNP allele associated with low fiber content in canola is determined.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein, except for the definitions, subject matter disclaimers or disavowals in such references. The references discussed herein are provided solely for their disclosure prior to the filing date of the present application. For any definitions, subject matter disclaimers or disavowals and to the extent that the incorporated material is inconsistent with the express disclosure herein, the language in the disclosure controls.

The following examples are included to demonstrate various embodiments of the invention and are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### EXAMPLES

### Example 1: Plant Materials and Identification of an ADF QTL

A DH population was developed from two black seeded elite canola lines. NS7627MC is a male spring canola type that provides low fiber content and high protein in the seed meal. G00178MC is also a male spring line that provides a commodity oil profile as well as low fiber content and high protein in the seed meal. The two lines differed by approximately 3 percent for ADF content, and approximately 2 percent in the protein content in the meal as measured by NIR, results shown in Table 1. 181 DH plants were generated and phenotyped using two different approaches. In the first approach, greenhouse (GH) grown seed from this population were subjected to NIR measurements for ADF and protein content in the meal; the same GH seed was also phenotyped using mass spectrometry (MS).

TABLE 1

| Line | Gender | PROTEIN | ADF |
|---|---|---|---|
| NS7627MC | Male | 49 | 16.7 |
| G00178MC | Male | 47.2 | 19.6 |

A high correlation (r=0.92) between NIR-generated values for ADF and MS-generated values for ADF was observed for the GH-based DH population. Each of the ADF traits also showed the expected negative correlation with protein measured in the GH-based DH population. Both ADF and protein displayed a normal distribution. Correlation results shown in Table 2 validate that the NIR method used was accurate and reliable for phenotyping ADF and protein content in meal.

TABLE 2

| | MS ADF | NIR ADF | Protein |
|---|---|---|---|
| MS ADF | 1 | | |
| NIR ADF | 0.92 | 1 | |
| Protein | −0.69 | −0.75 | 1 |

The second phenotyping approach involved growing the same DH population in fields at two separate locations in Manitoba and Saskatoon. Field grown seed was subjected to NIR, and phenotypic data analyzed by Best Linear Unbiased Predictions (BLUP) algorithm to account for spatial, year effects and location variability. BLUP values generated were used as phenotypes for trait mapping. The DH population's 181 individuals were genotyped using 10,800 Illumina XT genotyping assays. Composite interval analyses were performed for QTL detection. Two ADF-quantitative trait loci (QTLs) were identified on chromosomes N7 and N15 using greenhouse phenotyping data. The ADF-QTL on N15 explained more than 50% of the phenotypic variance for ADF in this population. Logarithm-of-odds method (LOD) indicated that the QTL on N15 is linked to the ADF phenotype with a very high degree of statistical significance. A threshold LOD score of at least 3 is typically used to establish high probability of genetic linkage. In these experiments, an LOD score of 31.6 indicated a very strongly correlation between N15 QTL and low ADF phenotype.

Meal ADF from the field grown seeds of the DH population ranged from 12.82 to 18.29; meal protein content from the same seeds ranged from 49.11 to 55.09. See Table 3. Ten DH lines, all of which carry the N15 QTL, were found to have both extremely low meal ADF profile (less than 14% ADF) and high meal protein content (greater than 53%).

TABLE 3

| | | ADF Field Grown | | | Protein Field Grown | | |
|---|---|---|---|---|---|---|---|
| | DH no. | Min | Max | Mean | Min | Max | Mean |
| N15_QTL | 98 | 12.82 | 16.73 | 14.55 | 49.11 | 55.09 | 52.53 |
| No N15_QTL | 73 | 14.07 | 18.29 | 16.03 | 50.51 | 53.57 | 51.95 |

The foregoing shows the identification of a previously unknown QTL on N15 that is strongly linked to low ADF phenotype.

### Example 2: High Resolution Mapping Indicate Deletions on Chr. N15

Genome wide association studies (GWAS) analyses were conducted to resolve the peak of the QTL and more precisely identify the relevant genomic region. The QTL region was narrowed down to approximately a 3 cM region (97.93-101.24). Surprisingly, within this 3 cM region, several physically linked Illumina XT SNP markers failed to produce amplification in approximately 50% of the individuals from the DH population, which suggested both Mendelian behavior and a potential segmental deletion. Markers that failed to produce an amplicon in a line are indicated by blank cell (-) in Table 4. Table 4 header indicates marker context sequence (SEQ ID) along with its genetic position in parenthesis; rows identify the wild-type or deletion line that was tested.

TABLE 4

| | SEQ ID | SEQ ID | SEQ ID | SEQ ID | SEQ ID | SEQ ID | SEQ ID | SEQ ID |
|---|---|---|---|---|---|---|---|---|
| | NO: 79 | NO: 72 | NO: 73 | NO: 74 | NO: 75 | NO: 76 | NO: 77 | NO: 78 |
| | (95.47) | (97.93) | (99.93) | (99.01) | (99.61) | (101.07) | (101.24) | (101.53) |
| Wild Type | G, G | G, G | T, T | G, G | T, T | G, G | C, C | C, C |
| 302 | G, G | — | — | — | — | — | — | C, C |
| 303 | G, G | — | — | — | — | — | — | C, C |
| 304 | G, G | — | — | — | — | — | — | C, C |
| 306 | G, G | — | — | — | — | — | — | C, C |
| 307 | G, G | — | — | — | — | — | — | C, C |
| G00178MC | G, G | — | — | — | — | — | — | C, C |
| NS7627MC | G, G | — | — | — | — | — | — | C, C |
| NS8245MC | G, G | — | — | — | — | — | — | C, C |

The same SNP marker assays also failed to produce amplicons when tested on small number of elite lines.

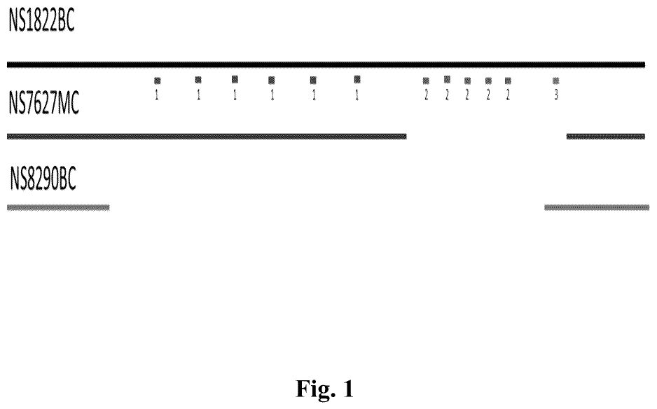

Although the size of the apparent deletion segments varied in different lines, including lines derived from NS8290BC, the deleted genomic segments overlapped based on the marker haplotype profile. The overlapping region for three lines is shown in FIG. 1 and Table 5. Table 5 shows Illumina XT marker sequence context from the region of N15 associated to low fiber/high protein.

TABLE 5

| | Wild Type | NS7627MC | NS8290BC |
|---|---|---|---|
| SEQ ID NO: 64 (86.06) | G, G | G, G | G, G |
| SEQ ID NO: 65 (86.09) | T, T | T, T | T, T |
| SEQ ID NO: 66 (86.58) | A, A | A, A | — |
| SEQ ID NO: 67 (87.54) | G, G | G, G | — |
| SEQ ID NO: 68 (89.77) | A, A | A, A | — |
| SEQ ID NO: 69 (92.05) | C, C | C, C | — |
| SEQ ID NO: 70 (93.18) | A, A | A, A | — |
| SEQ ID NO: 71 (93.76) | C, C | C, C | — |
| SEQ ID NO: 72 (97.93) | G, G | — | — |
| SEQ ID NO: 73 (98.93) | T, T | — | — |
| SEQ ID NO: 74 (99.01) | G, G | — | — |
| SEQ ID NO: 75 (99.61) | T, T | — | — |
| SEQ ID NO: 76 (101.07) | G, G | — | — |
| SEQ ID NO: 77 (101.24) | C, C | — | C, C |
| SEQ ID NO: 78 (101.53) | C, C | C, C | C, C |

Eleven XT markers fail to amplify in NS8290BC and six XT markers fail to amplify in NS7627MC. Marker group 1 amplifies in NS7627MC but not NS8290BC, group 2 does not amplify in neither NS7627MC nor NS8290BC, group 3 amplifies in NS8290BC but not in NS7627MC (see FIG. 1).

The foregoing results demonstrate the discovery of an unexpected segmental deletion on chromosome N15 at the locus that strongly correlates with a low ADF phenotype.

### Example 3: Genomic Characterization and Confirmation of N15 Deletions

Two complimentary approaches were employed to validate the presence of N15 segmental deletion described in Example 2. In one approach, short-read whole genome re-sequencing data was generated via Illumina sequencing platform for two elite lines: NS7627MC and NS8290BC that contain the N15 low fiber locus and the deletion at around 97.93-101.24 cM, as indicated by marker haplotype profile. Short-read sequences from these two lines were aligned to another, using wild-type proprietary reference genome NS1822BC as control, and the alignments were visualized using Integrated Genomics Viewer (IGV) software. Robinson et al. (2011) Nature Biotechnology, 29(1): 24-26. A segment of about 575 kb in the control NS1822BC genome has no corresponding short-read sequence matches on the N15 chromosome from NS7627MC genome. Similarly, about 1.65 Mb of N15 NS1822BC genome has no corresponding short-sequence match in NS8290BC genome.

In a second approach, two platinum quality reference genomes were developed for these elite lines NS7627MC and NS8290BC. The N15 target region around this genomic region was compared to control NS1822BC genome that does not contain the N15 locus. Comparative sequence analyses between these three genomes confirmed the deletion of a genomic segment of 598 kb in NS7627MC (corresponding to a deletion of chromosome N15 physical positions 52,639,876 to 53,229,532 of Darmor V10 reference genome). Further analyses revealed that the deleted region in NS7267MC was replaced by an approximately 518 kb insertion segment. See Table 6. About 239 kb of this insertion comprises sequence matches to chromosome N5, while the remaining insertion sequence has high similarity to several other B. napus chromosomes and could not be designated as a duplication from a single chromosome. Since the corresponding N5 homoeologous segment in NS7627MC is intact, it is unlikely that a simple N5 to N15 one-way non-reciprocal translocation occurred. Similarly, the corresponding deletion segment in NS8290BC was replaced by an approximately 1.1 Mb insertion segment. Approximately 580 kb of this second insertion segment includes sequence matches to N5 homoeologous chromosome, while the remaining has high similarity to several other B. napus chromosomes and could not be designated as a duplication from a single chromosome. This indicates a deletion on N15, followed by replacement with duplicated segments of N5 in an unknown mechanism, as one possible explanation. Wild type (comprising 100 bp upstream and 100 bp downstream) sequence corresponding to each of the deletion break points is provided herein as follows: NS7627MC deletion start point (position 101 of SEQ ID NO:80); NS7627MC deletion end point (position 101 of SEQ ID NO:81); NS8290BC start point (position 101 of SEQ ID NO:82); NS8290BC end point (position 101 of SEQ ID NO:83). Table 6 shows the physical position of the genomic start and end points that correspond to the deleted segment of N15; Table 6 also indicates the length of the replacement segment (insertion) that has replaced the N15 deleted segment in NS7627MC and NS8290BC.

TABLE 6

| | Deletion | Deletion | |
|---|---|---|---|
| | Segment Start | Segment End | Length |
| Genome | Position | Position | (bp) |
| Darmor V10 | 52639876 | 53229532 | 589656 |
| NS7627MC | 54183221 | 54700755 | 517534 |
| | (SEQ ID NO: | (SEQ ID NO: | (insertion) |
| | 80; nt. 101) | 81; nt. 101) | |
| Darmor V10 | 51500326 | 53185850 | 1685524 |
| NS8290BC | 52373865 | 53464890 | 1091025 |
| | (SEQ ID NO: | (SEQ ID NO: | (insertion) |
| | 82; nt. 101) | 83; nt. 101) | |

The foregoing results confirm the presence of N15 genomic deletion and provide detailed structural characterization of the deletion on chromosome N15 that strongly correlates with a low ADF phenotype.

### Example 4: N15 Deletion and Wild Type Marker Development

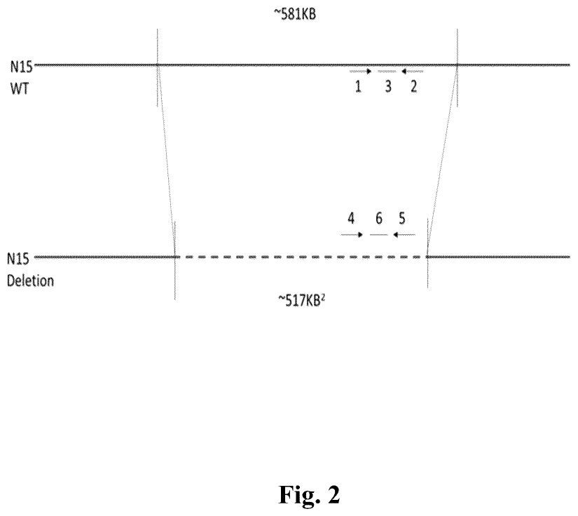

High throughput co-dominant assays for detecting the presence absence of N15 deletion variants and the zygosity state of N15 deletion locus were designed. Primer and probe sequences were created for the purpose of amplifying and detecting DNA sequences within the wild type N15 sequence that is present in wild type lines (e.g., NS1822BC), but is deleted in NS7627MC and/or NS8290BC—these are examples of an N15 wild type assay (“WT Assay”). Primer and probe sequences were also designed to amplify and detect DNA sequences within the insertion segment (at the N15 deletion locus) in NS7627MC and/or NS8290BC—these are examples of an N15 deletion assay (DEL Assay). In these examples, probe for detecting wild type N15 segment (indicating no deletion) was tagged with a VIC fluorophore and the probe for detecting the insertion segment (i.e., indicating N15 segment deletion) in the NS7627MC DEL Assay was tagged with a FAM fluorophore. These tags are detectable using TaqMan assay chemistry. See FIG. 2, which is a schema showing relative locations of forward primers, reverse primers and probes that can be used to assay the presence, absence, and/or zygosity of the N15 deletion.

Because the WI Assay is designed to detect the segment of N15 found in most, if not all, wild type lines (e.g. NS1822BC) but absent in lines having the disclosed N15 deletion, the wild-type marker can be used to detect (by absence of fluorophore signal) the presence of N15 deletion in lines such as NS7627MC, NS8290BC, or other lines having an N15 deletion.

The WI Assay and DEL Assay primers and probes are designed so that they can be combined in a single PCR amplification reaction to simultaneously detect all zygosity states of the N15 deletion locus in tested canola samples.

Table 7 provides a list of primers and probes that can be used in DEL Assay to detect the presence/absence of the N15 deletion (e.g., in NS7627MC) or WI Assay to detect the presence/absence of wild type lines in which the corresponding N15 segment is intact (not deleted).

TABLE 7

| SEQUENCE ID # | DESCRIPTION |
|---|---|
| SEQ ID NO: 1 | NS7627MC Deletion Probe |
| SEQ ID NO: 2 | NS7627MC Deletion Probe |
| SEQ ID NO: 3 | NS7627MC Deletion Probe |
| SEQ ID NO: 4 | NS7627MC Deletion Probe-Production marker |
| SEQ ID NO: 5 | NS7627MC Deletion Probe |
| SEQ ID NO: 6 | NS7627MC Deletion Probe |
| SEQ ID NO: 7 | NS7627MC WT Probe-Production Marker |
| SEQ ID NO: 8 | NS7627MC WT Probe |
| SEQ ID NO: 9 | NS7627MC WT Probe |
| SEQ ID NO: 10 | NS7627MC WT Probe |
| SEQ ID NO: 11 | NS7627MC WT Probe |
| SEQ ID NO: 12 | NS7627MC WT Probe |
| SEQ ID NO: 13 | NS7627MC Deletion Forward Primer |
| SEQ ID NO: 14 | NS7627MC Deletion Forward Primer |
| SEQ ID NO: 15 | NS7627MC Deletion Forward Primer |
| SEQ ID NO: 16 | NS7627MC Deletion Forward Primer-Production Marker |
| SEQ ID NO: 17 | NS7627MC Deletion Forward Primer |
| SEQ ID NO: 18 | NS7627MC Deletion Forward Primer |
| SEQ ID NO: 19 | NS7627MC WT Forward Primer-Production Marker |
| SEQ ID NO: 20 | NS7627MC WT Forward Primer |
| SEQ ID NO: 21 | NS7627MC WT Forward Primer |
| SEQ ID NO: 22 | NS7627MC WT Forward Primer |
| SEQ ID NO: 23 | NS7627MC WT Forward Primer |
| SEQ ID NO: 24 | NS7627MC WT Forward Primer |
| SEQ ID NO: 25 | NS7627MC Deletion Reverse Primer |
| SEQ ID NO: 26 | NS7627MC Deletion Reverse Primer |
| SEQ ID NO: 27 | NS7627MC Deletion Reverse Primer |
| SEQ ID NO: 28 | NS7627MC Deletion Reverse Primer-Production Marker |
| SEQ ID NO: 29 | NS7627MC Deletion Reverse Primer |
| SEQ ID NO: 30 | NS7627MC Deletion Reverse Primer |
| SEQ ID NO: 31 | NS7627MC WT Reverse Primer-Production Marker |
| SEQ ID NO: 32 | NS7627MC WT Reverse Primer |
| SEQ ID NO: 33 | NS7627MC WT Reverse Primer |
| SEQ ID NO: 34 | NS7627MC WT Reverse Primer |
| SEQ ID NO: 35 | NS7627MC WT Reverse Primer |
| SEQ ID NO: 36 | NS7627MC WT Reverse Primer |
| SEQ ID NO: 37 | NS7627MC Deletion Probe |
| SEQ ID NO: 38 | NS7627MC Deletion Probe |
| SEQ ID NO: 39 | NS7627MC Deletion Probe |
| SEQ ID NO: 40 | NS7627MC WT Probe |
| SEQ ID NO: 41 | NS7627MC WT Probe |
| SEQ ID NO: 42 | NS7627MC WT Probe |
| SEQ ID NO: 43 | NS7627MC WT Probe |
| SEQ ID NO: 44 | NS7627MC WT Probe |
| SEQ ID NO: 45 | NS7627MC WT Probe |
| SEQ ID NO: 46 | NS7627MC Deletion Forward Primer |
| SEQ ID NO: 47 | NS7627MC Deletion Forward Primer |
| SEQ ID NO: 48 | NS7627MC Deletion Forward Primer |
| SEQ ID NO: 49 | NS7627MC WT Forward Primer |
| SEQ ID NO: 50 | NS7627MC WT Forward Primer |
| SEQ ID NO: 51 | NS7627MC WT Forward Primer |
| SEQ ID NO: 52 | NS7627MC WT Forward Primer |
| SEQ ID NO: 53 | NS7627MC WT Forward Primer |
| SEQ ID NO: 54 | NS7627MC WT Forward Primer |
| SEQ ID NO: 55 | NS7627MC Deletion Reverse Primer |
| SEQ ID NO: 56 | NS7627MC Deletion Reverse Primer |
| SEQ ID NO: 57 | NS7627MC Deletion Reverse Primer |
| SEQ ID NO: 58 | NS7627MC WT Reverse Primer |
| SEQ ID NO: 59 | NS7627MC WT Reverse Primer |
| SEQ ID NO: 60 | NS7627MC WT Reverse Primer |
| SEQ ID NO: 61 | NS7627MC WT Reverse Primer |
| SEQ ID NO: 62 | NS7627MC WT Reverse Primer |
| SEQ ID NO: 63 | NS7627MC WT Reverse Primer |
| SEQ ID NO: 64 | N101HTW-001 Illumina probe |
| SEQ ID NO: 65 | N101HTV-001 Illumina probe |
| SEQ ID NO: 66 | N101HTU-001 Illumina probe |
| SEQ ID NO: 67 | N101HTA-001 Illumina probe |
| SEQ ID NO: 68 | N002GHT-001 Illumina probe |
| SEQ ID NO: 69 | N101HMX-001 Illumina probe |
| SEQ ID NO: 70 | N100B31-001 Illumina probe |
| SEQ ID NO: 71 | N101HM4-001 Illumina probe |
| SEQ ID NO: 72 | N05301-1 Illumina probe |
| SEQ ID NO: 73 | N04729-1 Illumina probe |
| SEQ ID NO: 74 | N002GHH-001 Illumina probe |
| SEQ ID NO: 75 | N101HHR-001 Illumina probe |
| SEQ ID NO: 76 | N101HHA-001 Illumina probe |
| SEQ ID NO: 77 | N101HH3-001 Illumina probe |
| SEQ ID NO: 78 | N101HGY-001 Illumina probe |
| SEQ ID NO: 79 | N101HJK-001 Illumina probe |
| SEQ ID NO: 80 | NS7627MC deletion segment left break point (100 bp 5′ |
| | and 100 bp 3′ flanking wild type corresponding sequence) |
| SEQ ID NO: 81 | NS7627MC deletion segment right break point (100 bp 5′ |
| | and 100 bp 3′ flanking wild type corresponding sequence) |
| SEQ ID NO: 82 | NS8290BC deletion segment left break point (100 bp 5′ |
| | and 100 bp 3′ flanking wild type corresponding sequence) |
| SEQ ID NO: 83 | NS8290BC deletion segment right break point (100 bp 5′ |
| | and 100 bp 3′ flanking wild type corresponding sequence) |

The foregoing provides components and methods for assays (DEL Assays) to detect the presence or absence of the N15 deletion disclosed herein as well as components and methods for assays (WT Assays) to detect the presence or absence of intact N15 genomic sequence (that is not deleted) at the same locus.

### Example 5: Use of N15 Deletion and Wild Type Markers to Screen Germplasm

A pool of elite proprietary Brassica napus lines from North America (NA), Europe (EU), Australia (AU) was genotyped with a DEL assay and a WT Assay marker from Example 4. None of the tested EU winter canola lines contained the N15 deletion from NS7627MC. By contrast, 17% of the NA germplasm and 2.5% of the Australian germplasm were found to contain this deletion. The zygosity state of individual lines was pooled to estimate the allele frequency of the NS7627MC N15 deletion in the elite lines from each geography, results shown in Table 8.

TABLE 8

| Elite Line | Homozygous | Heterozygous | Homozygous | Total No. | N15 Deletion |
|---|---|---|---|---|---|
| Origin | N15 Deletion | N15 Deletion | Wild Type | of Lines | Frequency |
| North | 61 | 1 | 294 | 356 | 17.42% |
| America | | | | | |
| Europe | 0 | 0 | 95 | 95 | 0.00% |
| Australia | 3 | 0 | 114 | 117 | 2.56% |
| Total (All) | 64 | 1 | 503 | 568 | 11.44% |

The foregoing shows that the compositions and assay methods disclosed herein can be used to detect the presence or absence of the N15 deletion in a naïve (i.e., not preselected) pool of elite Brassica napus lines.

1. A method for selecting a Brassica napus plant, germplasm, or grain thereof that comprises a low fiber content trait, the method comprising:
a. obtaining a nucleic acid sample from one or more Brassica napus plants, germplasms, or grains;
b. screening each sample for genomic sequence comprising a deleted genomic segment of Brassica napus chromosome N15 (N15 deletion), wherein the N15 deletion is located between genomic positions corresponding to SEQ ID NO:65 (N101HTV-001) and SEQ ID NO:78 (N101HGY-001) and wherein the N15 deletion is associated with low fiber content in Brassica napus; and
c. selecting one or more of the plants, germplasms, or grains that comprises the N15 deletion.

2. The method of claim 1, wherein the method comprises screening each sample for the absence of one or more markers in genomic sequence located between N15 chromosome genomic position 86.09 cM and position 101.53 cM.

3.-4. (canceled)

5. The method of claim 1, wherein the N15 deletion segment is at least about 1,667 kb in length.

6. The method of claim 1, wherein the method comprises screening each sample for the absence of one or more markers in the N15 chromosomal region between genomic positions corresponding to SEQ ID NO:82 and SEQ ID NO:81, or between SEQ ID NO:80 and SEQ ID NO:81, or between SEQ ID NO:82 and SEQ ID NO:83, or between SEQ ID NO:80 and SEQ ID NO:83 respectively.

7. The method of any one of claim 1, wherein the method comprises screening each sample for the presence of insertion sequence that has replaced the deleted genomic segment on chromosome N15.

8. The method of claim 7, wherein the method comprises screening each sample for insertion of replacement sequence between a deletion start break point and deletion end break point located within genomic positions corresponding to those shown in SEQ ID NO:82 and SEQ ID NO:81, respectively; or within SEQ ID NO:80 and SEQ ID NO:81, respectively; or within SEQ ID NO:82 and SEQ ID NO:83, respectively; or within SEQ ID NO:80 and SEQ ID NO:83, respectively.

9. The method of claim 1, wherein the method comprises screening each sample by nucleotide sequencing.

10. (canceled)

11. A method of selecting a Brassica napus plant, germplasm, or grain thereof that comprises a low fiber content trait, the method comprising:
a. obtaining a nucleic acid sample from one or more Brassica napus plants, germplasms, or grains thereof, each sample being representative of a plurality of plants, germplasms, or grains;
b. screening each representative sample for genomic sequence comprising a deleted genomic segment of Brassica napus chromosome N15 (NIS deletion), wherein the N15 deletion is located between genomic positions corresponding to SEQ ID NO: 65 (N101HTV-001) and SEQ ID NO:78 (N101HGY-001) and wherein the NIS deletion is associated with low fiber content in Brassica napus; and
c. selecting one or more of the plurality of plants, germplasms, or grains thereof, wherein the representative sample for each selected plurality is identified as having the N15 deletion.

12. The method of claim 11, wherein the representative sample is a sample of grain and the method further comprises producing meal from the selected plurality of grains.

13. The method of claim 11, further comprising preparing canola meal from one or more of the selected plants, germplasms, or grains.

14. A method for producing a Brassica napus plant comprising a low fiber content trait, the method comprising:
a. isolating or providing a nucleic acid sample from each of one or more Brassica napus plants, grains, or germplasm thereof;
b. screening each sample for a nucleic acid comprising a deleted genomic segment of Brassica napus chromosome N15 (N15 deletion), wherein the N15 deletion is located between sequences SEQ ID NO:65 (N101HTV-001) and SEQ ID NO: 79 (N101HGY-001) and wherein the N15 deletion is associated with low fiber content in Brassica napus;
c. selecting a first Brassica napus plant, grain, or germplasm thereof identified as having the N15 deletion; and
d. crossing the first plant selected in step c. with a second plant to produce progeny plants, wherein at least one of the progeny plants comprises the one or more low fiber content marker alleles.

15. The method of claim 14, wherein the method screening the sample for the absence of one or more markers in genomic sequence located between N15 chromosome genomic position 86.09 cM and position 101.53 cM.

16.-17. (canceled)

18. The method of claim 14, wherein the N15 deletion segment is at least about 1,667 kb in length.

19. The method of any of one of claim 14, wherein the method comprises screening the sample for the absence of one or more markers in the N15 chromosomal region between genomic positions corresponding to SEQ ID NO:82 and SEQ ID NO:81, respectively; or between SEQ ID NO:80 and SEQ ID NO:81, respectively; or between SEQ ID NO:82 and SEQ ID NO:83, respectively; or between SEQ ID NO:80 and SEQ ID NO:83, respectively.

20. The method of any one of claim 14, wherein the method comprises screening the sample for the presence of insertion sequence that has replaced the deleted genomic segment on chromosome N15.

21. The method of any one of claim 15, wherein the method comprises screening the sample by nucleotide sequencing.

22. The method of any one of claim 15, wherein the screening comprises amplifying and or sequencing from 10 to 300 bases upstream and/or downstream of one of the N15 deleted segment break points to thereby detect the N15 deletion.

23. A method of introducing a low fiber content trait into a Brassica plant comprising:
i. crossing a first parent Brassica plant comprising the N15 deletion with a second parent plant that does not comprise the N15 deletion to produce one or more hybrid progeny plants;
ii. obtaining a nucleic acid sample from one or more hybrid progeny plants; and
iii. selecting one or more hybrid progeny plants having the N15 deletion in accordance with method of claim 1.

24. The method of claim 23, further comprising:
d. crossing the one or more selected progeny plants with the first or second parent Brassica plant (the recurrent parent plant) to produce backcross progeny plants;
e. obtaining a nucleic acid sample from one or more backcross progeny plants; and
f. selecting the one or more backcross progeny plants having the N15 deletion to produce another generation of backcross progeny plants.

25. The method of claim 24, further comprising:
g. repeating steps (d), (e), and (f) three or more times to produce backcross progeny plants that comprise the N15 deletion and the agronomic characteristics of the recurrent parent plant when grown in the same environmental conditions.
